(19) United States
(12) Patent Application Publication
(10) Pub. No.: US 2018/0301223 A1
(43) Pub. Date: Oct. 18, 2018

(54) Advanced Tensor Decompositions For Computational Assessment And Prediction From Data

(71) Applicant: University of Utah Research Foundation, Salt Lake City, UT (US)

(72) Inventor: Orly Alter, Salt Lake City, UT (US)

(21) Appl. No.: 15/566,298

(22) PCT Filed: Apr. 14, 2016
(86) PCT No.: PCT/US16/27642

§ 371 Date: Oct. 13, 2017

Related U.S. Application Data
(60) Provisional application No. 62/147,555, filed on Apr. 14, 2015.
(60) Provisional application No. 62/147,545, filed on Apr. 14, 2015.

(51) Int. Cl. G16H 50/20; G06F 19/18

(52) U.S. Cl. CPC G16H 50/20; G06F 19/18

(57) ABSTRACT

Data can be characterized and compared by applying an unfolding algorithm to each of at least two N^{th} order tensors, representing the data, to generate at least two matrices, wherein N>2. The at least two tensors can have a matching number of columns in each of all dimensions except an N^{th} dimension. The applying the unfolding algorithm preserves the number of columns in one dimension common to (a) one of the at least two tensors and (b) a corresponding one of the at least two matrices, wherein each of the at least two matrices is a full column rank matrix. Each of the matrices is a unique, weighted sum of subtensors having a matching number of columns in each of all dimensions, at least two of the sums having different weighting coefficients. A relative significance of the subtensors is determined as a ratio of the weighting coefficients.

### CROSS-REFERENCE TO RELATED APPLICATIONS

This application claims the benefit of the priority of U.S. Provisional Application No. 62/147,555, entitled “Advanced Tensor Decompositions for Computational Assessment and Prediction from Data,” and U.S. Provisional Application No. 62/147,545, entitled “Genetic Alterations in Ovarian Cancer,” each filed on Apr. 14, 2015, the disclosures of which are hereby incorporated by reference in their entirety.

### GOVERNMENT LICENSE RIGHTS

This invention was made with government support under DMS0847173 and HG004302 awarded by National Science Foundation and National Institutes of Health. The government has certain rights in this invention.

### FIELD

The subject technology relates generally to computational assessment and prediction from data.

### BACKGROUND

In many areas of science, especially in biotechnology, the number of high-dimensional datasets recording multiple aspects of a single phenomenon is increasing. This increase is accompanied by a fundamental need for mathematical frameworks that can compare multiple large-scale matrices with different row dimensions. Some of these areas may involve disease prediction based on biological data related to patient and normal samples.

For example, glioblastoma multiforme (GBM), the most common malignant brain tumor in adults, is characterized by poor prognosis. GBM tumors may exhibit a range of copy-number alterations (CNAs), many of which play roles in the cancer's pathogenesis. Large-scale gene expression and DNA methylation profiling efforts have identified GBM molecular subtypes, distinguished by small numbers of biomarkers. However, the best prognostic predictor for GBM remains the patient's age at diagnosis.

### SUMMARY

According to some embodiments, the subject technology provides frameworks that can simultaneously compare and contrast two datasets arranged in large-scale tensors of the same column dimensions but with different row dimensions in order to find the similarities and dissimilarities among them. According to some embodiments, a tensor generalized singular value decomposition (tGSVD), described herein, is an exact, unique, simultaneous decomposition for comparing and contrasting two tensors of arbitrary order.

According to some embodiments, the matrix GSVD and the matrix higher-order GSVD (HO GSVD) are limited to datasets arranged in matrices, i.e., second-order tensors. Exact and unique simultaneous decomposition for two tensors can be performed to generalize the matrix GSVD to a tensor GSVD by following steps analogous to these that generalize the matrix SVD to the tensor, or higher-order SVD (HOSVD). This tensor GSVD transforms two tensors of the same numbers of columns across, e.g., the x- and the y-axes, and different numbers of rows across the z-axes, into weighted sums of “subtensors,” where each subtensor is an outer product of one x-, one y- and one z-axis vector. The sets of x-, y- and z-axes vectors are computed by using the matrix GSVD of the two tensors unfolded along their corresponding axes. This is different from previous tensor GSVDs, which, e.g., do not use the GSVD in the computation of each of the sets of vectors. From the GSVD it follows that a different set of orthogonal basis vectors Uᵢ is computed for each of the two tensors Tᵢ across the z-axes, with a one-to-one correspondence among these vectors. The sets of basis vectors across the x- and y-axes, Vₓ and V_{y}, are identical for both tensor factorizations, and are not, in general, orthogonal:

$${T}_{i} = {ℛ}_{i} \times {}_{z}^{}{U}{}_{i}^{} \times {}_{x}^{}{V}{}_{x}^{} \times {}_{y}^{}{V}{}_{y}^{} = {\sum }_{a} {\sum }_{b} {\sum }_{c} {r}_{i , abc} {𝒮}_{i} ( a , b , c ) , \\ {𝒮}_{i} ( a , b , c ) = {U}_{i , a} ⊗ {V}_{x , b} ⊗ {V}_{y , c} , i = 1 , 2.$$

To enable the interpretation of this tensor GSVD, the significance of the subtensor S₁(a, b, c) in T₁ is defined relative to that of the corresponding subtensor S₂(a, b, c) in T₂ in terms of an “angular distance” that is a function of the ratio of the weighting coefficients r_{1,abc} and r_{2,abc}. This angular distance is a function of the generalized singular values that correspond to U₁ and U₂ only, and is independent of the values that correspond to either Vₓ or V_{y}. The matrix GSVD and the tensor HOSVD are special cases of this tensor GSVD.

According to some embodiments, a method for characterization of data includes applying a decomposition algorithm, by a processor, to Nth-order tensors [symbol] and [symbol] representing data, wherein N>2 and wherein tensors [symbol] and [symbol] have matching number of columns in all dimensions except an n^{th} dimension, to generate, for each of the tensors, a weighted sum of a set of subtensors, the sets of subtensors having one-to-one correspondence and the sums having different weighting coefficients. A relative significance of the subtensors is determined as the ratio of the weighting coefficients. The data can include indicators, represented in respective rows and columns of the tensors, of values of at least two index parameters. According to some embodiments, an indicator of a health parameter of a subject is determined based on the relative significance of the subtensors.

Applying the decomposition algorithm comprises unfolding each of the tensors along the n^{th} dimension to generate, for each of the tensors, a basis vector corresponding to the n^{th} dimension values preserved by the unfolding. Each of the subtensors can be or include an outer product of vectors from every dimension of the corresponding tensor

The tensor GSVD (tGSVD) can be used to transform tensor [symbol] and a tensor [symbol] into weighted sums of subtensors. Vectors in the tensor [symbol] along an n^{th} index into a tensor GSVD (tGSVD) can be appended. Vectors in the tensor [symbol] along an n^{th} index into the tGSVD can also be appended.

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1, clause 13, or clause 15. The other clauses can be presented in a similar manner.

Clause 1. A method, for characterization of data, comprising:
applying an unfolding algorithm, by a processor, to each of at least two Nth order tensors, representing data, to generate at least two matrices, wherein N>2, wherein the at least two tensors have a matching number of columns in each of all dimensions except an Nth dimension, wherein the applying the unfolding algorithm preserves the number of columns in one dimension common to (a) one of the at least two tensors and (b) a corresponding one of the at least two matrices, wherein each of the at least two matrices is a full column rank matrix, wherein each of the matrices is a unique, weighted sum of subtensors having a matching number of columns in each of all dimensions, at least two of the sums having different weighting coefficients;
determining a relative significance of the subtensors as a ratio of the weighting coefficients;
determining and outputting, by a processor and based on the relative significance of the subtensors, an indicator of a health parameter of a subject, wherein the health parameter comprises at least one of a differential diagnosis, a first health status of the subject, a disease subtype, at least one of an estimated probability or an estimated risk of a second health status of the subject, an indicator of a prognosis of the subject, or a predicted response to a treatment of the subject.

Clause 2. The method of clause 1, wherein the tensors have one-to-one mappings among the columns across all but the Nth dimension of each of the tensors.

Clause 3. The method of clause 1, wherein the tensors do not have one-to-one mappings among the rows across the Nth dimension of each of the tensors.

Clause 4. The method of clause 1, further comprising applying a decomposition algorithm, by a processor, to the at least two subtensors, to generate, from the at least two subtensors A and B, eigenvectors of each of AAT, ATA, BBT, and BTB.

Clause 5. The method of clause 1, wherein the data comprises indicators, represented in respective rows and columns of the tensor, of values of at least two index parameters.

Clause 6. The method of clause 1, wherein the applying the unfolding algorithm includes appending into (N−1)th order tensors into (N−2)th order tensors that span (N−2) dimensions in each tensor.

Clause 7. The method of clause 1, wherein the applying the unfolding algorithm includes appending into a matrix the columns or rows across a preserved dimension in each tensor.

Clause 8. The method of clause 1, wherein each subtensor is an outer product of one x-, one y- and one z-axis vector.

Clause 9. The method of clause 8, wherein the sets of x-, y- and z-axes vectors are computed by using a matrix GSVD of the tensors unfolded along their corresponding axes.

Clause 10. The method of clause 1, further comprising, based on the indicator of the health parameter of the subject, applying a treatment to the subject.

Clause 11. The method of clause 10, wherein the treatment comprises administering a drug to the subject, admitting the subject to a care facility, or performing an operation on the subject.

Clause 12. The method of clause 1, wherein the tensors are generated by folding a plurality of matrices into the tensors.

Clause 13. A method, for characterization of data, comprising:
receiving, an indicator of a health parameter of a subject, wherein the health parameter comprises at least one of a differential diagnosis, a first health status of the subject, a disease subtype, at least one of an estimated probability or an estimated risk of a second health status of the subject, an indicator of a prognosis of the subject, or a predicted response to a treatment of the subject;
based on the indicator of the health parameter of the subject, applying a treatment to the subject;
wherein the indicator is determined by:
   applying an unfolding algorithm, by a processor, to each of at least two Nth order tensors, representing data, to generate at least two matrices, wherein N>2, wherein the at least two tensors have a matching number of columns in each of all dimensions except an Nth dimension, wherein the applying the unfolding algorithm preserves the number of columns in one dimension common to (a) one of the at least two tensors and (b) a corresponding one of the at least two matrices, wherein each of the at least two matrices is a full column rank matrix, wherein each of the matrices is a unique, weighted sum of subtensors having a matching number of columns in each of all dimensions, at least two of the sums having different weighting coefficients;
   determining a relative significance of the subtensors as a ratio of the weighting coefficients;
   determining, based on the relative significance of the subtensors, the indicator.

Clause 14. The method of clause 13, wherein the treatment comprises administering a drug to the subject, admitting the subject to a care facility, or performing an operation on the subject.

Clause 15. A system, for characterization of data, comprising:
an unfolding module configured to apply an unfolding algorithm, by a processor, to each of at least two Nth order tensors, representing data, to generate at least two matrices, wherein N>2, wherein the at least two tensors have a matching number of columns in each of all dimensions except an Nth dimension, wherein the applying the unfolding algorithm preserves the number of columns in one dimension common to (a) one of the at least two tensors and (b) a corresponding one of the at least two matrices, wherein each of the at least two matrices is a full column rank matrix, wherein each of the matrices is a unique, weighted sum of subtensors having a matching number of columns in each of all dimensions, at least two of the sums having different weighting coefficients;
a first determining module configured to determine a relative significance of the subtensors as a ratio of the weighting coefficients;
a second determining module configured to determine, by a processor and based on the relative significance of the subtensors, an indicator of a health parameter of a subject, wherein the health parameter comprises at least one of a differential diagnosis, a first health status of the subject, a disease subtype, at least one of an estimated probability or an estimated risk of a second health status of the subject, an indicator of a prognosis of the subject, or a predicted response to a treatment of the subject;
an outputting module, configured to output the indicator.

Clause 16. The system of clause 15, wherein the tensors have one-to-one mappings among the columns across all but the Nth dimension of each of the tensors.

Clause 17. The system of clause 15, wherein the tensors do not have one-to-one mappings among the rows across the Nth dimension of each of the tensors.

Clause 18. The system of clause 15, further comprising applying a decomposition algorithm, by a processor, to the at least two subtensors, to generate, from the at least two subtensors A and B, eigenvectors of each of AAT, ATA, BBT, and BTB.

Clause 19. The system of clause 15, wherein the data comprises indicators, represented in respective rows and columns of the tensor, of values of at least two index parameters.

Clause 20. The system of clause 15, wherein the applying the unfolding algorithm includes appending into (N−1)th order tensors into (N−2)th order tensors that span (N−2) dimensions in each tensor.

Clause 21. The system of clause 15, wherein the applying the unfolding algorithm includes appending into a matrix the columns or rows across a preserved dimension in each tensor.

Clause 22. The system of clause 15, wherein each subtensor is an outer product of one x-, one y- and one z-axis vector.

Clause 23. The system of clause 22, wherein the sets of x-, y- and z-axes vectors are computed by using a matrix GSVD of the tensors unfolded along their corresponding axes.

Clause 24. The system of clause 15, further comprising, based on the indicator of the health parameter of the subject, applying a treatment to the subject.

Clause 25. The system of clause 24, wherein the treatment comprises administering a drug, admitting the subject to a care facility, or performing an operation on the subject.

Clause 26. The system of clause 15, wherein the tensors are generated by folding a plurality of matrices into the tensors.

Additional features and advantages of the subject technology will be set forth in the description below, and in part will be apparent from the description, or may be learned by practice of the subject technology. The advantages of the subject technology will be realized and attained by the structure particularly pointed out in the written description and claims hereof as well as the appended drawings.

It is to be understood that both the foregoing general description and the following detailed description are exemplary and explanatory and are intended to provide further explanation of the subject technology as claimed.

### BRIEF DESCRIPTION OF THE DRAWINGS

The accompanying drawings, which are included to provide further understanding of the subject technology and are incorporated in and constitute a part of this description, illustrate aspects of the subject technology and, together with the specification, serve to explain principles of the subject technology.

FIG. 1 is a high-level diagram illustrating examples of tensors including biological datasets, according to some embodiments.

FIG. 2 is a high-level diagram illustrating a linear transformation of three-dimensional arrays, according to some embodiments.

FIG. 3 is a block diagram illustrating a biological data characterization system coupled to a database, according to some embodiments.

FIG. 4 is a flowchart of a method for disease related characterization of biological data, according to some embodiments.

FIG. 5 shows a matrix of higher-order tensors, according to some embodiments of the subject technology.

FIG. 6 shows how a tensor GSVD generalizes the matrix GSVD from two matrices to two higher-order tensors, in analogy, but not in equivalent mathematical formulation, to the tensor HOSVD's generalization of the matrix SVD, according to some embodiments of the subject technology.

FIG. 7 shows a tGSVD that has become the GSVD in the matrix limit, according to Corollary 1, according to some embodiments of the subject technology described herein.

FIG. 8 shows a tGSVD that has become the HOSVD in the limit where one tensor has ones on the diagonal and zeros everywhere else, according to Corollary 2, according to some embodiments of the subject technology described herein.

FIG. 9 shows GSVD of patient-matched but probe-independent GBM tumor and normal datasets. Raster display, with relative copy-number gain (red), no change (black) and loss (green). The significance of a pattern from VT, or “probelet,” in the tumor dataset relative to its significance in the normal dataset is defined in terms of an “angular distance” that is a function of the ratio of the pattern's significance in each dataset individually (i.e., the fraction of total information that the pattern contains). This is depicted in the bar chart display, where angular distances above 2π/9 represent tumor-exclusive patterns and those below −π/6 represent normal-exclusive patterns.

FIGS. 10A, 10B, and 10C show survival analyses of TCGA OV patients classified by tensor GSVD (FIG. 10A), tumor stage at diagnosis (FIG. 10B), and both (FIG. 10C).

FIG. 11 is a simplified diagram of a system, in accordance with various embodiments of the subject technology.

FIG. 12 is a block diagram illustrating an exemplary computer system with which a client device and/or a server of FIG. 11 can be implemented.

### DETAILED DESCRIPTION

In the following detailed description, specific details are set forth to provide an understanding of the subject technology. It will be apparent, however, to one ordinarily skilled in the art that the subject technology may be practiced without some of these specific details. In other instances, well-known structures and techniques have not been shown in detail so as not to obscure the subject technology. U.S. Provisional Application No. 61/553,840, entitled “Genomic Tensor Analysis for Medical Assessment and Prediction,” was filed on Oct. 31, 2011 and published on Mar. 14, 2013 as WO 2013/036874. U.S. Provisional Application No. 61/553,870, entitled “Genetic Alterations in Glioblastoma,” was filed on Oct. 31, 2011 and published on May 10, 2013 as WO 2013/067050. The technical subject matter of U.S. Provisional Application Nos. 61/553,840 and 61/553,870, and the corresponding publications, WO 2013/036874 and WO 2013/067050, are hereby incorporated by reference in their entirety.

According to some embodiments, the subject technology provides frameworks that can simultaneously compare and contrast two datasets arranged in large-scale tensors of the same column dimensions but with different row dimensions in order to find the similarities and dissimilarities among them. According to some embodiments, a tensor GSVD (tGSVD), described herein, is an exact, unique, simultaneous decomposition for comparing and contrasting two tensors of arbitrary order.

As used herein, script letters (e.g. [symbol] ) are used to denote tensors, capital letters (e.g. A) to indicate matrices, and lower case letters (e.g. a) to represent scalars. The exception is for indices, where i,j or a, b, c are typically used. The maximum for an index is given by I. The index of the n^{th} axis is iₙ and n has maximum value N. For an N-dimensional tensor, the indices are given as i₁ to i_{N}. Also, the entry in the i^{th} row and j^{th} column of the matrix A is denoted aᵢⱼ. When talking about multidimensional tensors, row is used to refer to the first dimension, whereas column is used for all others.

The subject technology can be applied to a variety of fields to analyze data used in an generated by entities within the field. Such fields include finance, advertising, medicine, biology, astronomy, among others. For example, subject technology may be applied to personalize medicine for analysis of DNA copy number, DNA methylation, mRNA expression, imaging, and medical records. By further example, the subject technology may be used to analyze, in medicine, a large number of high-dimensional datasets, recording multiple aspects of a disease across the same set of patients, such as in The Cancer Genome Atlas (TCGA).

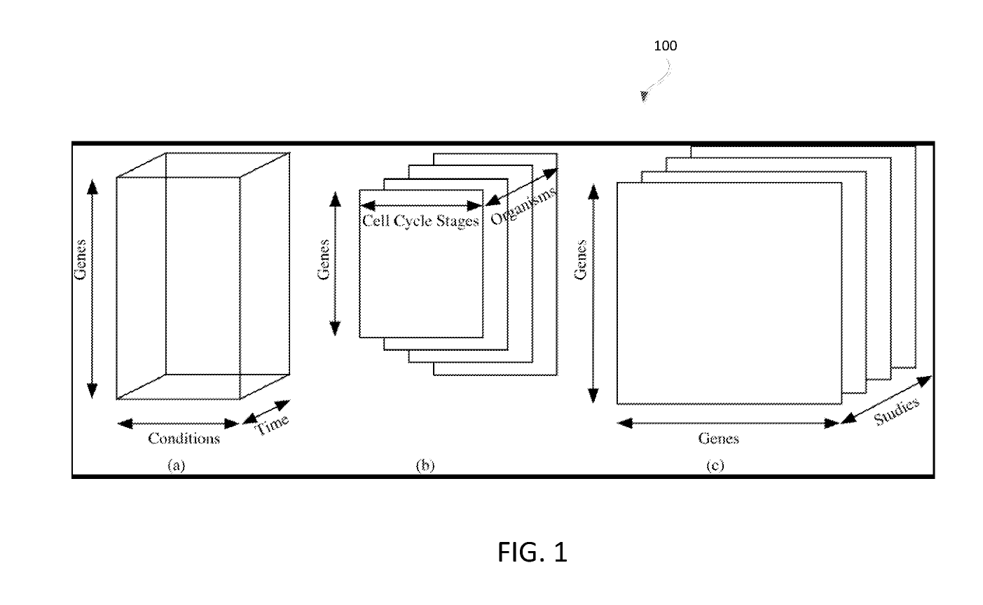

FIG. 1 is a high-level diagram illustrating examples of tensors 100 including biological datasets, according to some embodiments. In general, a tensor representing a number of biological datasets may comprise an Nth-order tensor including a number of multi-dimensional (e.g., two or three dimensional) matrices. The Nth-order tensor may include a number of biological datasets. Some of the biological datasets may correspond to one or more biological samples. Some of the biological dataset may include a number of biological data arrays, some of which may be associated with one or more subjects. Some examples of biological data that may be represented by a tensor includes tensors (a), (b) and (c) shown in FIG. 1. The tensor (a) represents a third order tensor (i.e., a cuboid), in which each dimension (e.g., gene, condition and time) represent a degree of freedom in the cuboid. If unfolded into a matrix, these degrees of freedom may be lost and most of the data included in the tensor may also be lost. However, decomposing the cuboid using a tensor decomposition technique, such as higher-order eigen-value decomposition (HOEVD) or higher-order single value decomposition (HOSVD) may uncover patterns of mRNA expression variations across the genes, the time points and conditions.

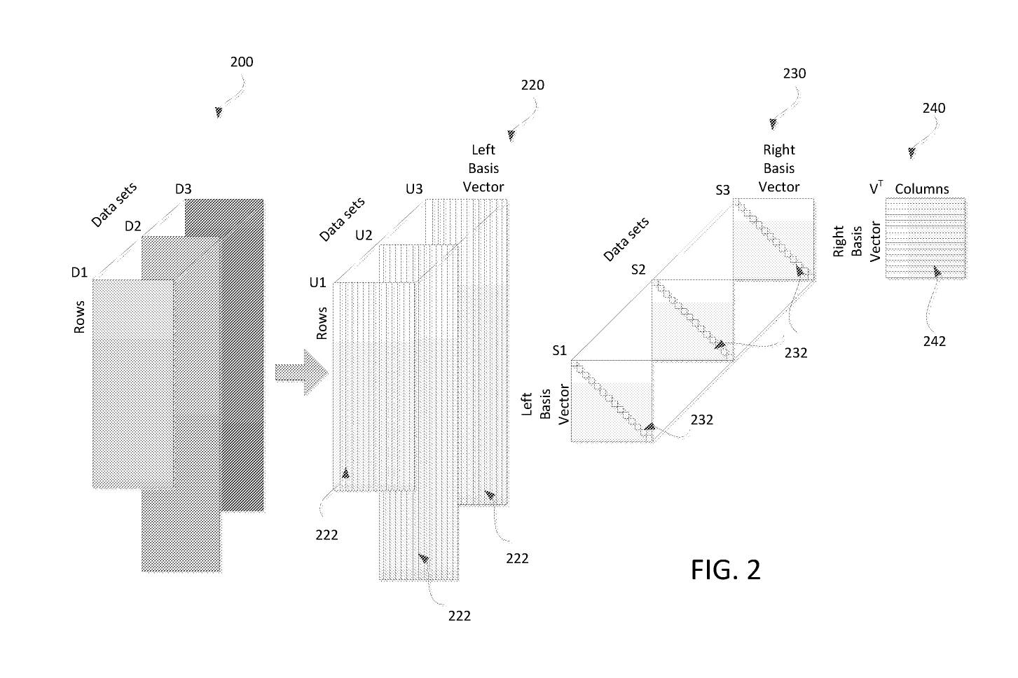

In the example tensor (b) the biological datasets are associated with genes and the one or more subjects comprises organisms and data arrays may include cell cycle stages. The tensor decomposition in this case may allow, for example, integrating global mRNA expressions measured for various organisms, removal of experimental artifacts and identification of significant combinations of patterns of expression variation across the genes, for various organisms and for different cell cycle stages. Similarly, in tensor (c) the biological datasets are associated with a network K of N-genes by N-genes. Where the network K may represent a number of studies on the genes. The tensor decomposition (e.g., HOEVD) in this case may allow, for example, uncovering important relations among the genes (e.g., pheromone-response-dependent relation or orthogonal cell-cycle-dependent relation). An example of a tensor represented by a three-dimensional array is discussed below with respect to FIG. 2.

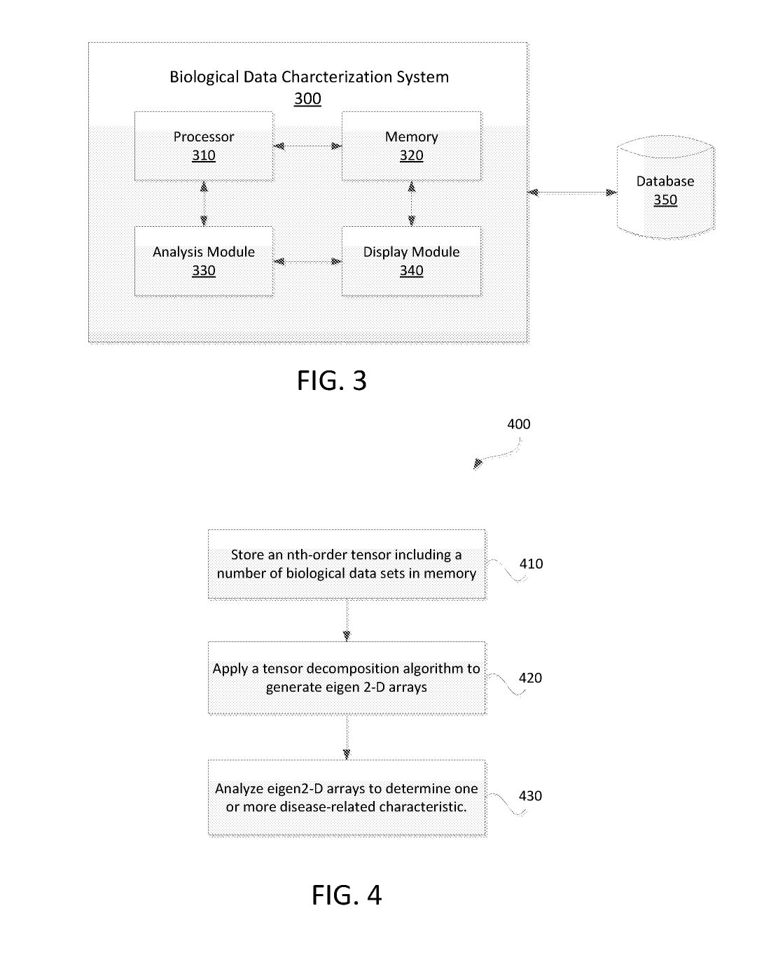

FIG. 2 is a high-level diagram illustrating a linear transformation of a number of two dimensional (2-D) arrays forming a three-dimensional (3-D) array 200, according to some embodiments. The 3-D array 200 may be stored in memory 300 (see FIG. 3). The 3-D array 200 may include a number N of biological datasets that correspond to genetic sequences. In some embodiments, the number N can be greater than two. Each biological dataset may correspond to a tissue type and can include a number M of biological data arrays. Each biological data array may be associated with a patient or, more generally, an organism). Each biological data array may include a plurality of data units (e.g., chromosomes). A linear transformation, such as a tensor decomposition algorithm may be applied to the 3-D array 200 to generate a plurality of eigen 2-D arrays 220, 230 and 240. The generated eigen 2-D arrays 220, 230 and 240 can be analyzed to determine one or more characteristics related to a disease (e.g., changes in glioblastoma multiforme (GBM) tumor with respect to normal tissue). The 3-D array 200 may comprise a number N of 2-D data arrays (D1, D2, D3, . . . DN) (for clarity only D1-D3 are shown in FIG. 2). Each of the 2-D data arrays (D1, D2, D3, . . . DN) can store one set of the biological datasets and includes M columns. Each column can store one of the M biological data arrays corresponding to a subject such as a patient.

As used herein, “health status” may refer to the presence, absence, quality, rank, or severity of any disease or health condition, history and physical examination finding, laboratory value, and the like. As used herein, a “health parameter” can include a differential diagnosis, meaning a diagnosis that is potential, confirmed, unconfirmed, based on a likelihood, ranked, or the like. A health parameter can include at least one of a differential diagnosis, a first health status of the subject, a disease subtype, an estimated probability, an estimated risk of a second health status of the subject, an indicator of a prognosis of the subject, or a predicted response to a treatment of the subject.

According to some embodiments, each biological data array may comprise biological data measurable by a DNA microarray (e.g., genomic DNA copy numbers, genome-wide mRNA expressions, binding of proteins to DNA and binding of proteins to RNA), a sequencing technology (e.g., using a different technology that covers the same ground as microarrays), a protein microarray or mass spectrometry, where protein abundance levels are measured on a large proteomic scale and a traditional measurement (e.g., immunohistochemical staining). The biological data may include chromatin or histone modification, a DNA copy number, an mRNA expression, a micro-RNA expression, a DNA methylation, binding of proteins to DNA, binding of proteins to RNA or protein abundance levels.

According to some embodiments, the biological data may be derived from a patient-specific sample including a normal tissue, a disease-related tissue or a culture of a patient's cell. The biological datasets may also be associated with genes and the one or more subjects comprises at least one of time points or conditions. The tensor decomposition of the Nth-order tensor may allow for identifying abnormal patterns to identify genes or proteins which enable including or excluding a diagnosis. Further, the tensor decomposition may allow classifying a patient into a subgroup of patients based on patient-specific genomic data, resulting in an improved diagnosis by identifying the patient's disease subtype. The tensor decomposition may also be advantageous in patients therapy planning, for example, by allowing patient-specific therapy to be designed based criteria, such as, a correlation between an outcome of a therapeutic method and a global genomic predictor.

In patients' disease prognosis, the tensor decomposition may facilitate designing at least one of predicting a patient's survival or a patient's response to a therapeutic method such as chemotherapy. The Nth-order tensor may include a patient's routine examination data, in which case decomposition of the tensor may allow designing of a personalized preventive regimen for a patient based on analyses of the patient's routine examinations data. According to some embodiments, the biological datasets may be associated with imaging data including magnetic resonance imaging (MM) data, electro cardiogram (ECG) data, electromyography (EMG) data or electroencephalogram (EEG) data. The biological datasets may be associated with vital statistics or phenotypic data.

According to some embodiments, the tensor decomposition of the Nth-order tensor may allow removing normal pattern copy number variations (CNVs) and an experimental variation from a genomic sequence. The tensor decomposition of the Nth-order tensor may permit an improved prognostic prediction of the disease by revealing disease-associated changes in chromosome copy numbers, focal copy number variations (CNVs) nonfocal CNVs and the like. The tensor decomposition of the Nth-order tensor may also allow integrating global mRNA expressions measured in multiple time courses, removal of experimental artifacts and identification of significant combinations of patterns of expression variation across the genes, the time points and the conditions.

According to some embodiments, applying the tensor decomposition algorithm may comprise applying at least one of a higher-order singular value decomposition (HOSVD), a higher-order generalized singular value decomposition (HO GSVD), a higher-order eigen-value decomposition (HOEVD) or parallel factor analysis (PARAFAC) to the Nth-order tensor. Some of the present embodiments apply HOSVD to decompose the 3-D array 200, as described in more detail herein. The PARAFAC method is known in the art and will not be described with respect to the present embodiments.

The HOSVD generated eigen 2-D arrays may comprise a set of N left-basis 2-D arrays 220. Each of the left-basis arrays 220 (e.g., U1, U2, U3, . . . UN) (for clarity only U1-U3 are shown in FIG. 2) may correspond to a tissue type and can include a number M of columns, each of which stores a left-basis vector 222 associated with a patient. The eigen 2-D arrays 230 comprise a set of N diagonal arrays (Σ1, Σ2, Σ3, . . . ΣN) (for clarity only Σ1-Σ3 are shown in FIG. 2). Each diagonal array (e.g., Σ1, Σ2, Σ3, . . . or ΣN) may correspond to a tissue type and can include a number N of diagonal elements 232. The 2-D array 240 comprises a right-basis array, which can include a number of right-basis vectors 242.

According to some embodiments, decomposition of the Nth-order tensor may be employed for disease related characterization such as diagnosing, tracking a clinical course or estimating a prognosis, associated with the disease.

FIG. 3 is a block diagram illustrating a data characterization system 300 coupled to a database 350, according to some embodiments. The system 300 includes a processor 310, memory 320, an analysis module 330 and a display module 340. Processor 310 may include one or more processors and may be coupled to memory 320. Memory 320 may comprise volatile memory such as random access memory (RAM) or nonvolatile memory (e.g., read only memory (ROM), flash memory, etc.). Memory 320 may also include machine-readable medium, such as magnetic or optical disks. Memory 320 may retrieve information related to the Nth-order tensors 100 of FIG. 1 or the 3-D array 200 of FIG. 2 from a database 350 coupled to the system 300 and store tensors 100 or the 3-D array 200 along with 2-D eigen-arrays 220, 230 and 240 of FIG. 2. Database 350 may be coupled to system 300 via a network (e.g., Internet, wide area network (WNA), local area network (LNA), etc.). According to some embodiments, system 300 may encompass database 350.

Processor 310 can apply a tensor decomposition algorithm, such as HOSVD, HO GSVD, or HOEVD to the tensors 100 or 3-D array 200 and generate eigen 2-D arrays 220, 230 and 240. According to some embodiments, processor 310 may apply the HOSVD or HO GSVD algorithms to array comparative genomic hybridization (aCGH) data from patient-matched normal and glioblastoma multiforme (GBM) blood samples. Application of HOSVD algorithm may remove one or more normal pattern copy number variations (CNVs) or experimental variations from the aCGH data. The HOSVD algorithm can also reveal GBM-associated changes in at least one of chromosome copy numbers, focal CNVs and unreported CNVs existing in the aCGH data. According to some embodiments, processor 310 may apply a decomposition algorithm to an Nth-order tensor representing data (N≥2) to generate, from two or more submatrices A and B of the tensor, eigenvectors of each of AA^{T}, A^{T}A, BB^{T}, and B^{T}B. The data may comprise indicators, represented in respective rows and columns of the tensor, of values of at least two index parameters. Analysis module 330 can perform disease related characterizations as discussed above. For example, analysis module 330 can facilitate various analyses of eigen 2-D arrays 230 of FIG. 2, for example, by assigning each diagonal element 232 of FIG. 2 to an indicator of a significance of a respective element of a right-basis vector 222 of FIG. 2, as described herein in more detail. According to some embodiments, Analysis module 330 can determine an indicator of a health parameter of a subject, based on the eigenvectors and on values, associated with the subject, of the two or more index parameters. The display module 240 can display 2-D arrays 220, 230 and 240 and any other graphical or tabulated data resulting from analyses performed by analysis module 330. Display module 330 can display the indicator of the health parameter of the subject in various ways including digital readout, graphical display, or the like. In embodiments, the indicator of the health parameter may be communicated, to a user or a printer device, over a phone line, a computer network, or the like. Display module 330 may comprise software and/or firmware and may use one or more display units such as cathode ray tubes (CRTs) or flat panel displays.

FIG. 4 is a flowchart of a method 400 for genomic prognostic prediction, according to some embodiments. Method 400 includes storing the N^{th}-tensors 100 of FIG. 1 or 3-D array 200 of FIG. 2 in memory 320 of FIG. 3 (410). A tensor decomposition algorithm such as HOSVD, HO GSVD, or HOEVD may be applied, by processor 310 of FIG. 3, to the datasets stored in tensors 100 or 3-D array 200 to generate eigen 2-D arrays 220, 230 and 240 of FIG. 2 (420). The generated eigen 2-D arrays 220, 230 and 240 may be analyzed by analysis module 330 to determine one or more disease-related characteristics (430). The HOSVD algorithm is mathematically described herein with respect to N>2 matrices (i.e., arrays D₁-D_{N}) of 3-D array 200. Each matrix can be a real mᵢ×n matrix. Each matrix is exactly factored as Dᵢ=Uᵢ ΣᵢV^{T}, where V, identical in all factorizations, is obtained from the balanced eigensystem SV=VΛ of the arithmetic mean S of all pairwise quotients AᵢAⱼ⁻¹ of the matrices Aᵢ=Dᵢ^{T} Di, where i is not equal to j, independent of the order of the matrices Dᵢ. It can be proved that this decomposition extends to higher orders all of the mathematical properties of the GSVD except for column-wise orthogonality of the matrices Ui (e.g., 2-D arrays 220 of FIG. 2).

It can be proved that matrix S is nondefective, i.e., S has n independent eigenvectors and that V is real and that the eigenvalues of S (i.e., λ₁, λ₂, . . . λ_{N}) satisfy λₖ≥1. In the described HO GSVD comparison of two matrices, the kₜₕ diagonal element of Σᵢ=diag (σ_{ι,k}) (e.g., the kₜₕ element 232 of FIG. 2) is interpreted in the factorization of the iₜₕ matrix D₁ as indicating the significance of the kₜₕ right basis vector vₖ in Dᵢ in terms of the overall information that vₖ captures in Dᵢ. The ratio σ_{ι,k}/σ_{j,k} indicates the significance of vₖ in Dᵢ relative to its significance in Dⱼ. It can also be proved that an eigenvalue λₖ=1 corresponds to a right basis vector vₖ of equal significance in all matrices Dᵢ and Dⱼ for all i and j, when the corresponding left basis vector u_{i,k} is orthonormal to all other left basis vectors in Uᵢ for all i. Detailed description of various analysis results corresponding to application of the HOSVD to a number of datasets related to patients and other subjects will be discussed below.

The matrix higher-order GSVD (HO GSVD) provides a framework that extends the GSVD by enabling a simultaneous decomposition of more than two such datasets, which by definition is exact and unique. The matrix HO GSVD for N≥2 matrices has been defined as Dᵢ∈ [symbol] ^{m}^{ᵢ}^{×n}, each with full column rank. Each matrix is exactly factored as Dᵢ=UᵢΣᵢV^{T}, where V, identical in all factorizations, is obtained from the eigensystem SV=VΛ of the arithmetic mean S of all pairwise quotients AᵢAⱼ⁻¹ of the matrices Aᵢ=Dᵢ^{T}Dᵢ, i≠j.

This decomposition extends to higher orders all of the mathematical properties of the GSVD except for complete column-wise orthogonality of the left basis vectors that form the matrix Uᵢ in each factorization. The matrix S is nondefective with V and Λ real. Its eigenvalues satisfy λₖ≥1. Equality holds if and only if the corresponding eigenvector υₖ is a right basis vector of equal significance in all matrices Dᵢ and Dⱼ, i.e., σ_{i,k}/σ_{j,k}=1 for all i and j, and the corresponding left basis vector u_{i,k} is orthogonal to all other vectors in Uᵢ for all i. The eigenvalues λₖ=1, therefore, define the “common matrix HO GSVD subspace.”

### Example 1

A HOSVD algorithm is mathematically described herein with respect to N>2 matrices (i.e., arrays D₁-D_{N}) of 3-D array 200. Each matrix can be a real mᵢ×n matrix. Each matrix is exactly factored as Dᵢ=UᵢΣᵢV^{T}, where V, identical in all factorizations, is obtained from the balanced eigensystem SV=VΛ of the arithmetic mean S of all pairwise quotients AᵢAⱼ⁻¹ of the matrices Aᵢ=Dᵢ^{T}Di, where i is not equal to j, independent of the order of the matrices Dᵢ. It can be proved that this decomposition extends to higher orders, all of the mathematical properties of the GSVD except for column-wise orthogonality of the matrices Uᵢ. It can be proved that matrix S is nondefective. In other words, S has n independent eigenvectors and that V is real and the eigenvalues of S (i.e., λ₁, λ₂, . . . λ_{N}) satisfy λₖ≥1.

In the described HO GSVD comparison of two matrices, the kth diagonal element of Σᵢ=diag(σ_{ι,k}) is interpreted in the factorization of the iₜₕ matrix Dᵢ as indicating the significance of the kₜₕ right basis vector vₖ in Dᵢ in terms of the overall information that vₖ captures in Dᵢ. The ratio σ_{ι,k}/σ_{j,k} indicates the significance of vₖ in Dᵢ relative to its significance in Dⱼ. It can also be proved that an eigenvalue λₖ=1 corresponds to a right basis vector vₖ of equal significance in all matrices Dᵢ and Dⱼ for all i and j when the corresponding left basis vector u_{i,k} is orthonormal to all other left basis vectors in Uᵢ for all i. Detailed description of various analysis results corresponding to application of the HOSVD to a number of datasets obtained from patients and other subjects will be discussed below.

A HOEVD tensor decomposition method can be used for decomposition of higher order tensors. Herein, as an example, the HOEVD tensor decomposition method is described in relation with a the third-order tensor of size K-networks×N-genes×N-genes as follows:

Let the third-order tensor {âₖ} of size K-networks×N-genes×N-genes tabulate a series of K genome-scale networks computed from a series of K genome-scale signals {êₖ}, of size N-genes×Mₖ-arrays each, such that âₖ=êₖêₖ^{T}, for all k=1, 2, . . . , K. We define and compute a HOEVD of the tensor of networks {âₖ},

$$\hat{a} ≡ {\sum }_{k = 1}^{K} {\hat{a}}_{k} = \hat{u} ( {\sum }_{k = 1}^{K} {\hat{ɛ}}_{k}^{2} ) {\hat{u}}^{T} = \hat{u} {\hat{ɛ}}^{2} {\hat{u}}^{T} , \qquad [ 5 ]$$

using the SVD of the appended signals ê≡(ê₁, ê₂, . . . , ê_{K})=û\hat{ε}\hat{v}^{T}, where the mth column of û, |αₘ [symbol] ≡û|m [symbol] , lists the genome-scale expression of the mth eigenarray of ê. Whereas the matrix EVD is equivalent to the matrix SVD for a symmetric nonnegative matrix, this tensor HOEVD is different from the tensor higher-order SVD (14-16) for the series of symmetric nonnegative matrices {âₖ}, where the higher-order SVD is computed from the SVD of the appended networks (â₁, â₂, . . . â_{K}) rather than the appended signals. This HOEVD formulates the overall network computed from the appended signals â=êê^{T} as a linear superposition of a series of M Σₖ₌₁^{K} Mₖ rank-1 symmetric “subnetworks” that are decorrelated of each other, â=Σₘ₌₁^{M} εₘ²|αₘ [symbol] [symbol] αₘ|. Each subnetwork is also decoupled of all other subnetworks in the overall network â, since \hat{ε} is diagonal.

This HOEVD formulates each individual network in the tensor {âₖ} as a linear superposition of this series of M rank-1 symmetric decorrelated subnetworks and the series of M(M−1)/2 rank-2 symmetric couplings among these subnetworks, such that

$${\hat{a}}_{k} = {\sum }_{m = 1}^{M} {ɛ}_{k , m}^{2} \vert {α}_{m} 〉 〈 {α}_{m} \vert + {\sum }_{m = 1}^{M} {\sum }_{l = m + 1}^{M} {ɛ}_{k , l m}^{2} ( \vert {α}_{l} 〉 〈 {α}_{m} \vert + \vert {α}_{m} 〉 〈 {α}_{l} \vert ) , \qquad [ 6 ]$$

for all k=1, 2, . . . , K. The subnetworks are not decoupled in any one of the networks {âₖ}, since, in general, {\hat{ε}ₖ²} are symmetric but not diagonal, such that ε_{k,lm}²≡ [symbol] l∥εₖ²∥m [symbol] = [symbol] m∥εₖ²∥l [symbol] ≠0. The significance of the mth subnetwork in the kth network is indicated by the mth fraction of eigen expression of the kth network ρ_{k,m}=ε_{k,m}²/(Σₖ₌₁^{K} Σₘ₌₁^{M} ε_{k,m}²)≥0, i.e., the expression correlation captured by the mth subnetwork in the kth network relative to that captured by all subnetworks (and all couplings among them, where Σₖ₌₁^{K} ε_{k,lm}²=0 for all 1≠m) in all networks. Similarly, the amplitude of the fraction ρ_{k,lm}=ε_{k,lm}²/(Σₖ₌₁^{K} Σₘ₌₁^{M} ε_{k,m}²) indicates the significance of the coupling between the lth and mth subnetworks in the kth network. The sign of this fraction indicates the direction of the coupling, such that ρ_{k,lm}>0 corresponds to a transition from the lth to the mth subnetwork and ρ_{k,lm}<0 corresponds to the transition from the mth to the metric distribution of the annotations among the N-genes and the subsets of n⊆N genes with largest and smallest levels of expression in this eigenarray. The corresponding eigengene might be inferred to represent the corresponding biological process from its pattern of expression.

For visualization, we set the x correlations among the X pairs of genes largest in amplitude in each subnetwork and coupling equal to ±1, i.e., correlated or anticorrelated, respectively, according to their signs. The remaining correlations are set equal to 0, i.e., decorrelated. We compare the discretized subnetworks and couplings using Boolean functions (6).

We parallel- and antiparallel-associate each subnetwork or coupling with most likely expression correlations, or none thereof, according to the annotations of the two groups of x pairs of genes each, with largest and smallest levels of correlations in this subnetwork or coupling among all X=N(N−1)/2 pairs of genes, respectively. The P value of a given association by annotation is calculated by using combinatorics and assuming hypergeometric probability distribution of the Y pairs of annotations among the X pairs of genes, and of the subset of y⊆Y pairs of annotations among the subset of x⊆X pairs of genes, P(x;y, Y, X)=(ₓ^{X})⁻¹ Σ_{z=y}^{x} (_{z}^{Y})(_{x-z}^{X-Y}), where (ₓ^{X})=X!x!⁻¹(X−x)⁻¹ is the binomial coefficient (17). The most likely association of a subnetwork with a pathway or of a coupling between two subnetworks with a transition between two pathways is that which corresponds to the smallest P value. Independently, we also parallel- and antiparallel-associate each eigenarray with most likely cellular states, or none thereof, assuming hypergeometric distribution of the annotations among the N-genes and the subsets of n⊆N genes with largest and smallest levels of expression in this eigenarray. The corresponding eigengene might be inferred to represent the corresponding biological process from its pattern of expression.

For visualization, we set the x correlations among the X pairs of genes largest in amplitude in each subnetwork and coupling equal to ±1, i.e., correlated or anticorrelated, respectively, according to their signs. The remaining correlations are set equal to 0, i.e., decorrelated. We compare the discretized subnetworks and couplings using Boolean functions (6).

With reference to FIG. 39 as shown in U.S. Published Application No. 2014/0303029, incorporated herein by reference, a higher-order EVD (HOEVD) of the third-order series of the three networks {â₁, â₂, â₃}. The network â₃ is the pseudoinverse projection of the network â₁ onto a genome-scale proteins' DNA-binding basis signal of 2,476-genes×12-samples of development transcription factors [3] (Mathematica Notebook 3 and Data Set 4), computed for the 1,827 genes at the intersection of â₁ and the basis signal. The HOEVD is computed for the 868 genes at the intersection of â₁, â₂ and â₃. Raster display of âₖ≈Σₘ₌₁³ ∈_{k,m}²|αₘ [symbol] [symbol] αₘ|+Σₘ₌₁³ Σₗ₌ₘ₊₁³ ∈_{k,lm}²(|αₗ [symbol] [symbol] αₘ|+|αₘ [symbol] [symbol] αₗ|), for all k=1, 2, 3, visualizing each of the three networks as an approximate superposition of only the three most significant HOEVD subnetworks and the three couplings among them, in the subset of 26 genes which constitute the 100 correlations in each subnetwork and coupling that are largest in amplitude among the 435 correlations of 30 traditionally-classified cell cycle-regulated genes. This tensor HOEVD is different from the tensor higher-order SVD [14-16] for the series of symmetric nonnegative matrices {â₁, â₂, â₃}. The subnetworks correlate with the genomic pathways that are manifest in the series of networks. The most significant subnetwork correlates with the response to the pheromone. This subnetwork does not contribute to the expression correlations of the cell cycle-projected network â₂, where ∈_{2,1}²≈0. The second and third subnetworks correlate with the two pathways of antipodal cell cycle expression oscillations, at the cell cycle stage G₁ vs. those at G₂, and at S vs. M, respectively. These subnetworks do not contribute to the expression correlations of the development-projected network â₃, where ∈_{3,2}²≈∈_{3,3}²≈0. The couplings correlate with the transitions among these independent pathways that are manifest in the individual networks only. The coupling between the first and second subnetworks is associated with the transition between the two pathways of response to pheromone and cell cycle expression oscillations at G₁ vs. those G₂, i.e., the exit from pheromone-induced arrest and entry into cell cycle progression. The coupling between the first and third subnetworks is associated with the transition between the response to pheromone and cell cycle expression oscillations at S vs. those at M, i.e., cell cycle expression oscillations at G₁/S vs. those at M. The coupling between the second and third subnetworks is associated with the transition between the orthogonal cell cycle expression oscillations at G₁ vs. those at G₂ and at S vs. M, i.e., cell cycle expression oscillations at the two antipodal cell cycle checkpoints of G₁/S vs. G₂/M. All these couplings add to the expression correlation of the cell cycle-projected â₂, where ∈_{2,12}², ∈_{2,13}², ∈_{2,23}²>0; their contributions to the expression correlations of â₁ and the development-projected â₃ are negligible (see also FIG. 4 of US 2014/0303029).

In embodiments, a tensor GSVD arranged in two higher-than-second-order tensors of matched column dimensions but independent row dimensions is used in the methods herein.

Primary OV tumor and normal DNA copy-number profiles of a set of 249 TCGA patients were selected. Each profile was measured in two replicates by the same set of two DNA microarray platforms. For each chromosome arm or combination of two chromosome arms, the structure of these tumor and normal discovery datasets [symbol] ₁ and [symbol] ₂, of K₁-tumor and K₂-normal probes×L-patients, i.e., arrays×M-platforms, is that of two third-order tensors with one-to-one mappings between the column dimensions L and M but different row dimensions K₁ and K₂, where K₁, K₂≥LM.

This tensor GSVD simultaneously separates the paired datasets into weighted sums of LM paired “subtensors,” i.e., combinations or outer products of three patterns each: Either one tumor-specific pattern of copy-number variation across the tumor probes, i.e., a “tumor arraylet” u_{1,a}, or the corresponding normal-specific pattern across the normal probes, i.e., the “normal arraylet” u_{2,a}, combined with one pattern of copy-number variation across the patients, i.e., an “x-probelet” v_{x,b}^{T} and one pattern across the platforms, i.e., a “y-probelet” v_{y,c}^{T}, which are identical for both the tumor and normal datasets,

$$\begin{matrix}{𝒟}_{i} = {R}_{i} \times {}_{a}^{}{U}{}_{i}^{} \times {}_{b}^{}{V}{}_{x}^{} \times {}_{c}^{}{V}{}_{y}^{} \\ = {\sum }_{a = 1}^{LM} {\sum }_{b = 1}^{L} {\sum }_{c = 1}^{M} {R}_{i , abc} {S}_{i} ( a , b , c )\end{matrix} \\ {S}_{i} ( a , b , c ) = {u}_{i , a} ⊗ {v}_{x , b}^{T} ⊗ {v}_{y , c}^{T} , i = 1 , 2 , \qquad ( 1 )$$

where ×ₐUᵢ, ×_{b}Vₓ and ×_{c}V_{y} denote tensor-matrix multiplications, which contract the LM-arraylet, L-x-probelet, and M-y-probelet dimensions of the “core tensor” [symbol] ᵢ with those of Uᵢ, Vₓ, and V_{y}, respectively, and where ⊗ denotes an outer product.

It was found that unfolding (or matricizing) both tensors [symbol] ᵢ into matrices, each preserving the K₁-row dimension, e.g., by appending the LM columns [symbol] _{i,:lm} of the corresponding tensor, gives two full column-rank matrices Dᵢ∈ [symbol] ^{k}^{ᵢ}^{×LM}. The column bases vectors Uᵢ were obtained from the GSVD of Dᵢ, i.e., the “row mode GSVD”

Dᵢ=( . . . , [symbol] _{i:lm}, . . . )=UᵢΣᵢV^{T}, i=1,2. (2)

Similarly, that unfolding both tensors [symbol] ᵢ into matrices, each preserving the L-x- (or M-y-) column dimension, e.g., by appending the KᵢM rows [symbol] _{i,k}_{ᵢ}_{:m}^{T} (or the KᵢL rows [symbol] _{i,k}_{ᵢ}_{l:}^{T}) of the corresponding tensor, gives two full column-rank matrices Dᵢₓ ∈ [symbol] ^{K}^{ᵢ}^{M×L} (or D_{iy} ∈ [symbol] ^{k}^{ᵢ}^{L×M}). We obtain the x- (or y-) row basis vectors Vₓ^{T} (or V_{y}^{T}), from the GSVD of Dᵢₓ (or D_{iy}), i.e., the x- (or y-) column mode GSVD,

Dᵢₓ=( . . . , [symbol] ᵢ^{T}_{k;m}, . . . )=UᵢₓΣᵢₓVₓ^{T},

D_{iy}=( . . . , [symbol] ᵢ^{T}_{k;l}, . . . )=U_{iy}Σ_{iy}V_{y}^{T}, i=1,2. (3)

Note that the x- and y-row bases vectors are, in general, non-orthogonal but normalized, and Vₓ and V_{y} are invertible. The column bases vectors are normalized and orthogonal, i.e., uncorrelated, such that Uᵢ^{T} Uᵢ=I.

Unfolding is performed on tensors of the same order, the tensors having one-to-one mappings among the columns across all but one the of corresponding dimensions among the tensors, but not necessarily among the rows across the one remaining dimension in each tensor. Each tensor is unfolded by, for N order tensors, preserving 1, 2, 3, . . . , N−2 dimensions, e.g., by appending into 2, 3, 4, . . . , N−1 order tensors the 1, 2, 3, . . . , N−2 order tensors that span these 1, 2, 3, . . . , N−2 dimensions in each tensor. For example, for third or higher-than-third order tensors, one of the dimensions is preserved, e.g., by appending into a matrix the columns or rows across that dimension in each tensor. By further example, for fourth or higher-than-fourth order tensors, two of the dimensions are preserved, e.g., by appending into a third-order tensor the matrices that span these two dimensions in each tensor. By further example, for fifth or higher order tensors, three of the dimensions are preserved. The unfolding can be full-column rank unfolding, wherein, for N order tensors, each of the N unfoldings preserves one dimension (e.g., by appending into a matrix the vectors that span each of these dimensions in each tensor) and produces a full-column rank matrix.

The generalized singular values are positive, and are arranged in Σᵢ, Σᵢₓ, and Σ_{iy} in decreasing orders of the corresponding “GSVD angular distances,” i.e., decreasing orders of the ratios σ_{1,a}/σ_{2,a}, σ_{1x,b}/σ_{2x,b}, and σ_{1y,c}/σ_{2y,c}, respectively. We then compute the core tensors [symbol] ᵢ by contracting the row-, x-, and y-column dimensions of the tensors [symbol] Dᵢ with those of the matrices Uᵢ, Vₓ⁻¹ and V_{y}⁻¹, respectively. For real tensors, the “tensor generalized singular values” [symbol] _{i,abc} tabulated in the core tensors are real but not necessarily positive. Our tensor GSVD construction generalizes the GSVD to higher orders in analogy with the generalization of the singular value decomposition (SVD) by the HOSVD, and is different from other approaches to the decomposition of two tensors.

It is proven herein that the tensor GSVD exists for two tensors of any order because it is constructed from the GSVDs of the tensors unfolded into full column-rank matrices (Lemma A Example 5). The tensor GSVD has the same uniqueness properties as the GSVD, where the column bases vectors u_{i,a} and the row bases vectors υ_{x,b}^{T} and u_{y,c}^{T} are unique, except in degenerate subspaces, defined by subsets of equal generalized singular values σᵢ, σᵢₓ, and σ_{iy}, respectively, and up to phase factors of ±1, such that each vector captures both parallel and antiparallel patterns. The tensor GSVD of two second-order tensors reduces to the GSVD of the corresponding matrices (see Example 5). The tensor GSVD of the tensor [symbol] ₁∈ [symbol] ^{LM×L×M}, which row mode unfolding gives the identity matrix D₁=I∈ [symbol] ^{LM×LM}, and a tensor [symbol] ₂ of the same column dimensions reduces to the HOSVD of [symbol] ₂ (Theorem A in Example 5).

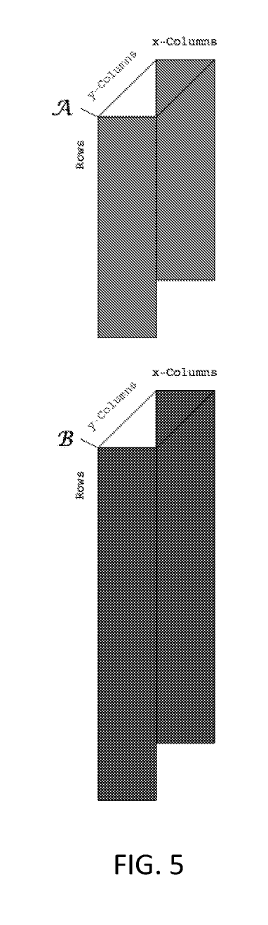

The significance of the subtensor Sᵢ(a, b, c) in the tensor [symbol] ᵢ is defined proportional to the magnitude of the corresponding tensor generalized singular values R_{i,abc} (FIG. 5), in analogy with the HOSVD,

P_{i,abc}=R_{i,abc}²/Σₐ₌₁^{LM}Σ_{b=1}^{L}Σ_{c=1}^{M}R_{i,abc}, i=1,2. (4)

The significance of S₁(a, b, c) in [symbol] ₁ relative to that of S₂(a, b, c) in [symbol] ₂ is defined by the “tensor GSVD angular distance” Θ_{abc} as a function of the ratio R_{1,abc}/R_{2,abc}. This is in analogy with, e.g., the row mode GSVD angular distance θₐ, which defines the significance of the column basis vector u_{i,a} in the matrix [symbol] ₁ of Eq. (2) relative to that of u_{2,a} in [symbol] ₂ as a function of the ratio σ_{1,a}/σ_{2,a},

Θ_{abc}=arctan(R_{1,abc}/R_{2,abc})−π/4,

θₐ=arctan(σ_{1,a}/σ_{2,a})−π/4. (5)

Because the ratios of the positive generalized singular values satisfy σ_{1,a}/σ_{2,a}∈[0, ∞), the row mode GSVD angular distances satisfy θₐ∈[−π/4, π/4]. The maximum (or minimum) angular distance, i.e., θₐ=π/4, which corresponds to σ_{1,a}/σ_{2,a}>>1 (or −π/4, which corresponds to σ_{1,a}/σ_{2,a}<<1), indicates that the row basis vector υ_{α}^{T} of Eq. (2), which corresponds to the column basis vectors u_{1,a} in [symbol] ₁ and u_{2,a} in [symbol] ₂, is exclusive to D₁ (or D₂). An angular distance of θₐ=0, which corresponds to σ_{1,a}/σ_{2,a}=1, indicates a row basis vector υ_{α}^{T} which is of equal significance in, i.e., common to both D₁ and D₂.

Thus, while the ratio σ_{1,a}/σ_{2,a} indicates the significance of u_{i,a} in D₁ relative to the significance of u_{2,a} in D₂, this relative significance is defined, as previously described, by the angular distance θₐ, a function of the ratio σ_{1,a}/σ_{2,a}, which is antisymmetric in D₁ and D₂. Note also that while other functions of the ratio σ_{1,a}/σ_{2,a} exist that are antisymmetric in D₁ and D₂, the angular distance θₐ, which is a function of the arctangent of the ratio, i.e., arctan(σ_{1,a}/σ_{2,a}) is the natural function to use, because the GSVD is related to the cosine-sine (CS) decomposition, as previously described, and, thus, σ_{1,a} and σ_{2,a} are related to the sine and the cosine functions of the angle θₐ, respectively.

Theorem 1.

The tensor GSVD angular distance equals the row mode GSVD angular distance, Θ_{abc}=θₐ.

Proof.

The unfolding of [symbol] ᵢ of Eq. (1) into Dᵢ of Eq. (2) unfolds the core tensors [symbol] ᵢ of Eq. (1) into matrices [symbol] ᵢ, which preserve the row dimensions, i.e., the LM-column bases dimensions of [symbol] ᵢ, and gives

Dᵢ=UᵢRᵢ(Vₓ^{T}⊗V_{y}^{T}

Rᵢ=(ΣᵢV^{T}(Vₓ⁻T⊗V_{y}^{T}), i=1,2, (6)

where ⊗ denotes a Kronecker product. Because Σᵢ are positive diagonal matrices, it follows that [symbol] _{1,abc}/ [symbol] _{2,abc}= [symbol] _{1,a}/ [symbol] _{2,a}=σ_{1,a}/σ_{2,a}. Substituting this in Eq. (5) gives Θ_{abc}=θₐ. Note that the proof holds for tensors of higher-than-third order.

From this it follows that the tensor GSVD angular distance |Θ_{abc}|≤π/4, and that, therefore, the ratio of the tensor generalized singular values [symbol] _{1,abc}/ [symbol] _{2,abc}>0, even though [symbol] _{1,abc} and [symbol] _{2,abc} are not necessarily positive. It also follows that Θ_{abc}=±π/4 indicate a subtensor exclusive to either [symbol] ₁ or [symbol] ₂, respectively, and that Θ_{abc}=0 indicates a subtensor common to both.

Note that in this embodiment since the generalized singular values are arranged in Σᵢ of Eq. (2) in a decreasing order of the row mode GSVD angular distances θₐ, the most tumor-exclusive tumor subtensors, i.e., S₁(a, b, c) where a maximizes θₐ of Eq. (5), correspond to a=1, whereas the most normal-exclusive normal sub-tensors, i.e., S₂(a, b, c) where a minimizes θₐ, correspond to a=LM.

Lemma A.

The tensor GSVD exists for any two, e.g., third-order tensors [symbol] ᵢ∈ [symbol] ^{K}^{ᵢ}^{×L×M} of the same column dimensions L and M but different row dimensions Kᵢ, where Kᵢ≥LM for i=1, 2, if the tensors unfold into full column-rank matrices, Dᵢ∈ [symbol] ^{K}^{ᵢ}^{×LM}, Dᵢₓ∈ [symbol] ^{K}^{ᵢ}^{M×L}, and D_{iy} ∈ [symbol] ^{K}^{ᵢ}^{L×M}, each preserving the Kᵢ-row dimension, L-x-, or M-y-column dimension, respectively.

Proof.

The tensor GSVD of Eq. (1), of the pair of third-order tensors [symbol] ᵢ, is constructed from the GSVDs of Eqs. (2) and (3), of the pairs of full column-rank matrices Dᵢ, Dᵢₓ, and D_{iy}, where i=1, 2. From the existence of the GSVDs of Eqs. (2) and (3) [5, 6], the orthonormal column bases vectors of Uᵢ, as well as the normalized x- and y-row bases vectors of the invertible Vₓ^{T} or V_{y}^{T}, exist, and, therefore, the tensor GSVD of Eq. (1) also exists. Note that the proof holds for tensors of higher-than-third order.

Lemma B.

The tensor GSVD has the same uniqueness properties as the GSVD.

Proof.

From the uniqueness properties of the GSVDs of Eqs. (2) and (3), the orthonormal column bases vectors u_{i,a}, and the normalized row bases vectors V_{x,b}^{T}, and V_{y,c}^{T} of the tensor GSVD of Eq. (1) are unique, except in degenerate subspaces, defined by subsets of equal generalized singular values σᵢ, σᵢₓ, and σ_{iy}, respectively, and up to phase factors of ±1. The tensor GSVD, therefore, has the same uniqueness properties as the GSVD. Note that the proof holds for tensors of higher-than-third order.

For two second-order tensors, the tensor GSVD reduces to the GSVD of the corresponding matrices. Proof. For two second-order tensors, e.g., the matrices Dᵢ∈ [symbol] ^{K}^{ᵢ}^{×L}, the tensor GSVD of Eq. (1) is

$$\begin{matrix}{D}_{i} = {R}_{i} {\times }_{a} {U}_{i} {\times }_{b} {V}_{x} \\ = {U}_{i} {R}_{i} {V}_{x}^{T}\end{matrix} \qquad ( A 1 )$$

The row- and x-column mode GSVDs of Eqs. (2) and (3) are identical, because unfolding each matrix Dᵢ while preserving either its Kᵢ-row dimension, or L-x-column dimension results in Dᵢ, up to permutations of either its columns or rows, respectively,

Dᵢ=UᵢΣᵢVₓ^{T}=Dᵢₓ, i−1,2. (A2)

From the uniqueness properties of the tensor GSVD of Eq. (A1), and the GSVDs of Eq. (A2) it follows that Rᵢ=Σᵢ, and that for two second-order tensors, i.e., matrices, the tensor GSVD is equivalent to the GSVD.

Theorem A.

The tensor GSVD of the tensor [symbol] ₁∈ [symbol] ^{LM×L×M}, which row mode unfolding gives the identity matrix D₁=I∈ [symbol] ^{LM×LM}, and a tensor [symbol] ₂ of the same column dimensions reduces to the HOSVD of [symbol] ₂.

Proof.

Consider the GSVD of Eq. (2), of the matrices D₁=I and D₂, as computed by using the QR decomposition of the appended D₁ and D₂, and the SVD of the block of the resulting column-wise orthonormal Q that corresponds to D₂, i.e., Q₂=U_{Q}_{₂}Σ_{Q}_{₂}V_{Q}_{₂}^{T},

$$[ \begin{matrix}\text{?} \\ \text{?}\end{matrix} ] = [ \begin{matrix}\text{?} \\ \text{?}\end{matrix} ] = QR = {[ \begin{matrix}{Q}_{1} \\ {Q}_{2}\end{matrix} ]}^{R} = [ \begin{matrix}{R}^{- 1} \\ \text{?}\end{matrix} ] \text{?} , \\ \text{?} \text{indicates text missing or illegible when filed} \qquad ( A3 )$$

where R is upper triangular and, therefore, invertible. Since Q is column-wise orthonormal, V_{Q}_{₂}^{T}, is orthonormal, and Σ_{Q}_{₂} is positive diagonal, it follows that

$$\begin{matrix}I = {Q}_{1}^{T} Q 1 + {Q}_{2}^{T} Q 2 \\ = {R}^{- T} {R}^{- 1} + ( {V}_{1} {\sum }_{Q 2}^{2} {V}_{Q 2}^{T} \\ = {( {V}_{Q 2}^{T} R )}^{- 1} + {( {V}_{Q 2}^{T} R )}^{- 1} + {\sum }_{Q 2}^{2} ,\end{matrix} \\ {( I - {\sum }_{Q 2}^{2} )}^{- 1} = ( {V}_{Q 2}^{T} R ) {( {V}_{{Q}_{2}}^{T} R )}^{T} , \qquad ( A 4 )$$

and that

$${( I - {\sum }_{{Q}_{2}}^{2} )}^{\frac{1}{2}} {V}_{{Q}_{2}}^{T}$$

R is orthonormal. The GSVD of Eq. (2) factors the matrix D₂ into a column-wise or-thonormal U_{Q}_{₂}, a positive diagonal

$${\sum }_{{Q}_{2}} {( I - {\sum }_{{Q}_{2}}^{2} )}^{- \frac{1}{2}}$$

and an orthonormal

$${( I - {\sum }_{{Q}_{2}}^{2} )}^{\frac{1}{2}} {V}_{{Q}_{2}}^{T} R ,$$

and is, therefore, reduced to the SVD of D₂.

This proof holds for the GSVDs of Eq. (3). This is because the x- and y-column unfoldings of the tensor [symbol] ᵢ∈ [symbol] LM×L×M, which row mode unfolding gives the identity matrix D₁=I∈ [symbol] ^{LM×LM}, gives

$${D}_{1 x} = \begin{matrix}[ \begin{matrix}I \\ ⋮ \\ I \\ 0 \\ ⋮ \\ 0\end{matrix} ] & \begin{matrix}\} M \\ \} M ( M - 1 )\end{matrix}\end{matrix} , \\ {D}_{1 y} = \begin{matrix}[ \begin{matrix}I \\ ⋮ \\ I \\ 0 \\ ⋮ \\ 0\end{matrix} ] & \begin{matrix}\} L \\ \} L ( L - 1 )\end{matrix}\end{matrix} . \qquad ( A 5 )$$

The GSVDs of Eqs. (2) and (3), of any one of the matrices D₁, D₁ₓ, or D_{1y} with the corresponding full column-rank matrices D₂, D₂ₓ, or D_{2y}, are, therefore, reduced to the SVDs of D₂, D₂ₓ, or D_{2y}, respectively.

The tensor GSVD of Eq. (1), where the orthonormal column bases vectors u_{2,a}, and the normalized row bases vectors v_{x,b}^{T}, and v_{y,c}^{T} in the factorization of the tensor [symbol] ₂ are computed via the SVDs of the unfolded tensor is, therefore, reduced to the HOSVD of [symbol] ₂ [25-27]. Note that the proof holds for tensors of higher-than-third order.

The “tensor generalized Shannon entropy” of each dataset,

$$0 \leq {d}_{i} = - {( 2 log LM )}^{- 1} {\sum }_{a = 1}^{LM} {\sum }_{b = 1}^{L} {\sum }_{c = 1}^{M} {𝒫}_{i , abc} log {𝒫}_{i , abc} \leq 1 , \\ i = 1 , 2 , \qquad ( A6 )$$

measures the complexity of each dataset from the distribution of the overall information among the different subtensors. An entropy of zero corresponds to an ordered and redundant dataset in which all the information is captured by a single subtensor. An entropy of one corresponds to a disordered and random dataset in which all subtensors are of equal significance.

### Example 2

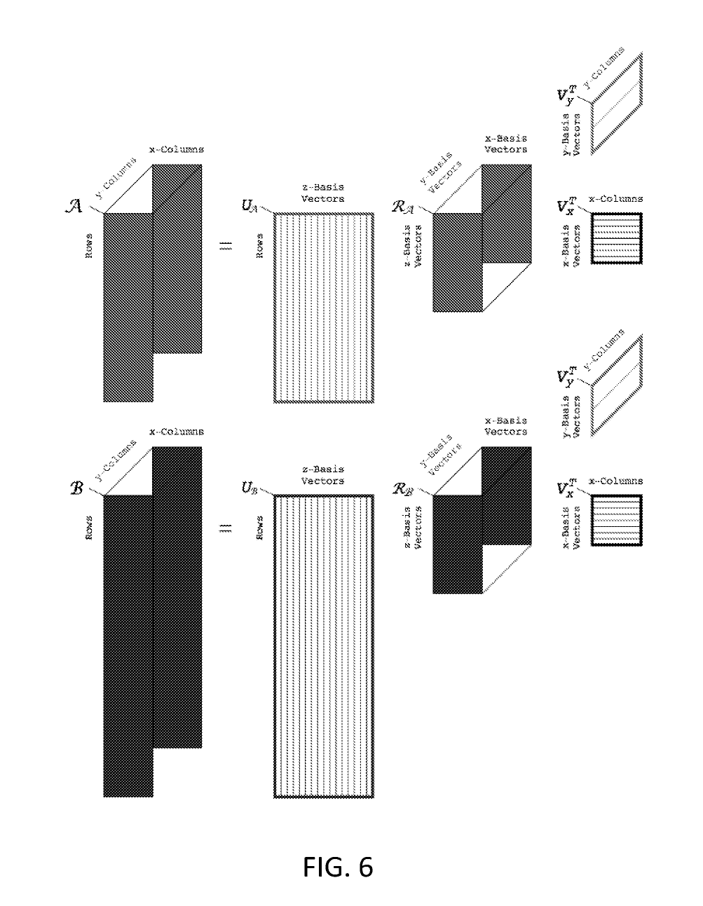

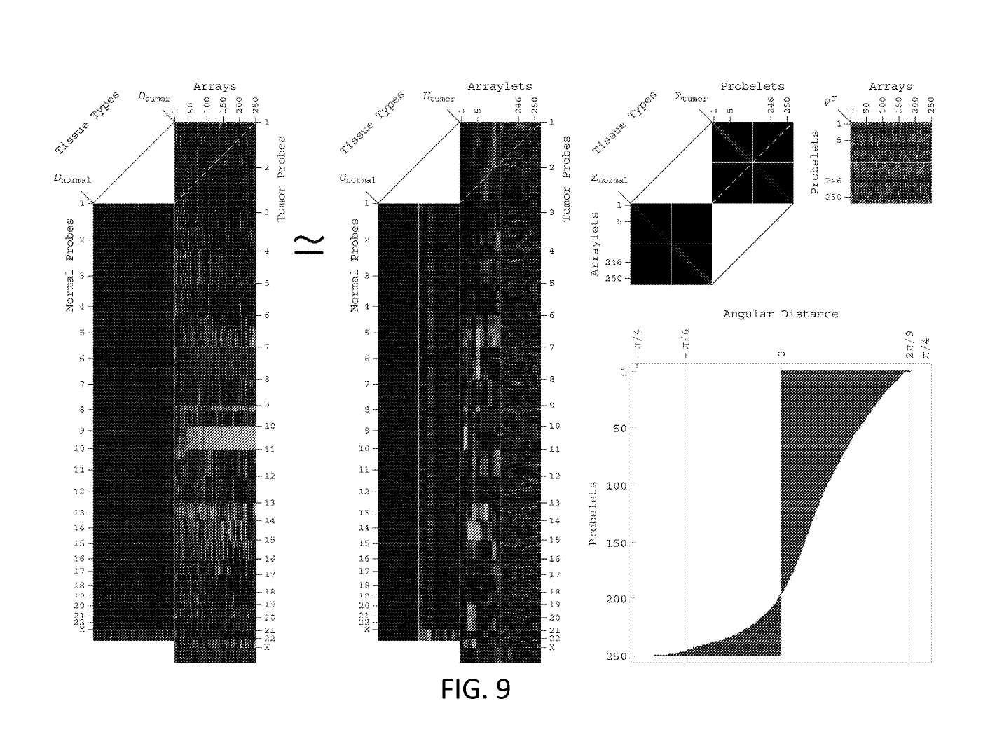

According to some embodiments, to define the tensor GSVD, the matrix GSVD generalized by following steps analogous to those that generalize the matrix SVD to a tensor SVD. The GSVD simultaneously decomposes two matrices of the same numbers of columns and different numbers of rows, as shown in FIG. 5, into unique, weighted sums of combinations of patterns of variation (see FIG. 9). A different set of orthogonal left basis vectors U_{A} and U_{B} is computed for each of the matrices A and B with a one-to-one correspondence among these vectors, as shown in FIG. 6. The Uᵢ(for i=A,B) matrices are column-wise orthonormal such that Uᵢ^{T}Uᵢ=I but UᵢUᵢ^{T}≠I in general. The set of right basis vectors V^{T} is identical for both matrix factorizations and the vectors are not, in general, orthogonal, but are normalized:

A=U_{A}Σ_{A}V^{T}=Σ_{A}×₁U_{A}×₂V

B=U_{B}Σ_{B}V^{T}=Σ_{B}×₁U_{B}×₂V

In analogy, a tensor GSVD for two tensors of the same numbers of columns across, e.g., the x- and the y-axes, and different numbers of rows across the z-axes, that transforms each of the two tensors into a unique, is defined as weighted sum of combinations of patterns of variation. In this case, each of the sets of patterns is computed by using the matrix GSVD of the two tensors unfolded along their corresponding axes. This decomposition transforms each of the two tensors into a unique, weighted sum of “subtensors,” where each subtensor is an outer product of one x-, one y- and one z-axis vector. The sets of x-, y- and z-axes vectors are computed by using the matrix GSVD of the two tensors unfolded along their corresponding axes. From the GSVD it follows that a different set of orthogonal basis vectors U_{A} and U_{B} is computed for each of the tensors A and B across the z-axes, with a one-to-one correspondence among these vectors (see FIG. 6). The Ui matrices are column-wise orthogonal such that Uᵢ^{T}Uᵢ=I but UᵢUᵢ^{T}≠I in general. The sets of vectors across the x- and y-axes Vₓ and V_{y} are identical for both tensor factorizations, and are not, in general, orthogonal. Thus, each of the tensors is rewritten as a weighted sum of subtensors S_{A}(a,b,c) and S_{B}(a,b,c) with the weighting coefficients R_{A,abc} and R_{B,abc}:

$$= {A}_{} {\times }_{1} {U}_{A} {\times }_{2} {V}_{x} {\times }_{3} {V}_{y} = {\sum }_{a} {\sum }_{b} {\sum }_{c} {R}_{A , abc} {S}_{A} ( a , b , c ) \\ = {B}_{} {\times }_{1} {U}_{B} {\times }_{2} {V}_{x} {\times }_{3} {V}_{y} = {\sum }_{a} {\sum }_{b} {\sum }_{c} {R}_{B , abc} {S}_{B} ( a , b , c )$$

where the subscript on the multiplication symbol indicates the axis for multiplication of a tensor by a matrix. As shown in FIG. 6, dimension one corresponds to the z-axis, two to the x-axis, and three to the y-axis. The core tensors, R_{A} and R_{B}, are full and non-negative. Additionally,

S_{A}(a,b,c)=U_{A,a}⊗V_{x,b}⊗V_{y,c}

S_{B}(a,b,c)=U_{B,a}⊗V_{x,b}⊗V_{y,c}

where the ⊗ symbol represents the outer product of vectors.

To enable the use of this tensor GSVD in the comparative modeling of two data tensors in order to find similarities and dissimilarities in the datasets, the significance of the subtensor S_{A}(a,b,c) in A relative to the significance of the corresponding subtensor S_{B}(a,b,c) in B is defined in terms of an angular distance that is a function of the ratio of the weighting coefficients R_{A,abc} and R_{B,abc.} This angular distance is a function of the generalized singular values corresponding to U_{A} and U_{B} only, and is independent of the generalized singular values corresponding to either Vₓ or V_{y}. The relative significance is defined as

θ=arctan(r_{A,i}/r_{B,i})−π/4

where r_{A,i} and r_{B,i} are corresponding elements of the core tensors, R_{A} and R_{B}. Values of θ closer to π/4 indicate that the corresponding pattern is exclusive to dataset A, whereas values close to −π/4 indicate exclusivity to dataset B. The ratio r_{A,i}/r_{B,i} is dependent only on the row (z-axis), and is invariant across other dimensions and therefore only depends on the GSVD of the first unfolding (preserving the z-axis) which is used to generate Uᵢ. Unfolding the tensor GSVD on the first axis gives,

A₍₁₎=U_{A}·R_{A,(1)}·(Vₓ⊗V_{y})^{T}

B₍₁₎=U_{B}·R_{B,(1)}·(Vₓ⊗V_{y})^{T}

where the ⊗ symbol represents the Kronecker product, i.e. the outer product of matrices, and the subscripts in parenthesis represent unfolding along the corresponding dimension. Performing the GSVD on A₍₁₎ and B₍₁₎ allows one to solve for the core tensors as,

R_{A,(1)}=Σ_{A}·W

R_{B,(1)}=Σ_{B}·W

where W is simply a matrix (identical in both equations) and Σ_{A} and Σ_{B} are the diagonal core matrices from the matrix GSVD. The matrix W cancels when dividing corresponding elements of R_{A} and R_{B} and the ratio of corresponding singular values from the matrix GSVD (σ_{A,i} and σ_{B,i}) remains:

$$\frac{{r}_{A , i}}{{r}_{B , i}} = \frac{{σ}_{A , i}}{{σ}_{B , i}}$$

### Example 3

Given two real tensors [symbol] ∈ [symbol] ^{I}^{_{1,}} ^{×I}^{₂}^{× . . . ×I}^{_{N}} and [symbol] ∈ [symbol] ^{I}^{_{1,}} ^{×I}^{₂}^{× . . . ×I}^{_{N}} that have full column rank when unfolded along each dimension, the tGSVD of [symbol] and [symbol] is

[symbol]

= [symbol] ×₁ [symbol] ×₂V₂ . . . ×_{N}V_{N}

[symbol]

= [symbol] ×₁ [symbol] ×₂V₂ . . . ×_{N}V_{N}

where [symbol] ∈ [symbol] ^{I}^{_{1,A}} ^{×I}^{₂}^{I}^{₃}^{. . . I}^{_{N}} and [symbol] ∈ [symbol] ^{I}^{_{1,B}} ^{×I}^{₂}^{I}^{₃}^{. . . I}^{_{N}} have orthonormal columns, Vₙ∈ [symbol] ^{I}^{ₙ}^{×I}^{ₙ} are nonsingular, and [symbol] , [symbol] ∈ [symbol] ^{I}^{₂}^{I}^{₃}^{. . . I}^{_{N}}^{×I}^{₂}^{×I}^{₃}^{× . . . ×I}^{_{N}} are the two core tensors and are generally full. The subscripts [symbol] and [symbol] distinguish non-identical entities corresponding to the tensors [symbol] and [symbol] , respectively. The notation Xₙ denotes multiplication of a tensor by a matrix on the n^{th} dimension.

According to some embodiments, the tGSVD is constructed by unfolding the tensors, computing the matrix GSVD (mGSVD), and saving the set of basis vectors corresponding to the dimension preserved by the unfolding. An unfolding of the tensor [symbol] along dimension n means appending the vectors of length Iₙ in [symbol] , i.e. those along n^{th} index, into a matrix. The mGSVD of [symbol] and [symbol] unfolded to preserve the n^{th} dimension is

A₍ₙ₎= [symbol] · [symbol] ·V⁽ⁿ⁾^{^{T}}

B₍ₙ₎= [symbol] · [symbol] ·V⁽ⁿ⁾^{^{T}}.

Where the subscript ₍ₙ₎ denotes unfolding along the n^{th} dimension, the superscript ⁽ⁿ⁾ indicates that the matrix corresponds to the n^{th} unfolding. From the properties of the mGSVD, [symbol] and [symbol] are column-wise orthogonal. [symbol] and [symbol] are diagonal, and V⁽ⁿ⁾^{^{T}} is invertible. The order in which the columns of A₍ₙ₎ and B₍ₙ₎ are unfolded does not affect the decomposition because the column vectors of [symbol] and [symbol] hold fundamental patterns from the column vectors of A₍ₙ₎ and B₍ₙ₎, which are independent of ordering in the matrices.

According to some embodiments, the tGSVD is constructed by setting [symbol] = [symbol] [symbol] = [symbol] , and Vₙ=V⁽ⁿ⁾ for 2≤n≤N. The core tensors, [symbol] and [symbol] , are then computed as

$$= {\times }_{1} {U}_{{( 1 )}^{T}}^{} {\times }_{2} {V}^{{( 2 )}^{- 1}} … {\times }_{N} {V}^{{( N )}^{- 1}}$$

$$= {\times }_{1} {U}_{{( 1 )}^{T}}^{} {\times }_{2} {V}^{( 2 ) - 1} … {\times }_{N} {V}^{( N ) - 1} .$$

The tGSVD can be reformulated so each of the tensors will be rewritten as a weighted sum of a set of subtensors, [symbol] _{(a, b, c)} and [symbol] _{(a, b, c)} for a third order tensor, with a one-to-one correspondence among these two sets of subtensors and with different weighting coefficients, [symbol] and [symbol] :

$$= {\sum }_{a} {\sum }_{b} {\sum }_{c} {r}_{, abc} ( a , b , c )$$

$$= {\sum }_{a} {\sum }_{b} {\sum }_{c} {r}_{, abc} ( a , b , c )$$

$$( a , b , c ) = {U}_{, a} ⊗ {V}_{2 , b} ⊗ {V}_{3 , c}$$

$$( a , b , c ) = {U}_{, a} ⊗ {V}_{2 , b} ⊗ {V}_{3 , c}$$

where the subscripts a, b, and c index column vectors of the matrices and ⊗ denotes an outer product of vectors.

Following from the existence of the mGSVD, existence of the tGSVD is shown in this lemma from its construction: Lemma 1 (Existence). For any two tensors, [symbol] and [symbol] , each with dimensionality N and matching number of columns in all dimensions except one (labeled as the first), there exists a decomposition of the form shown above given that the dimensions of the tensors satisfy the relationship

[symbol]

>I₂I₃ . . . I_{N}

[symbol]

>I₂I₃ . . . I_{N}

and for n≥2,

[symbol]

< [symbol] . . . Iₙ₋₁Iₙ₊₁ . . . I_{N}

[symbol]

< [symbol] I₂ . . . Iₙ₋₁Iₙ₊₁ . . . I_{N}.

Lemma 2 (Uniqueness).

Given the method of construction, the matrices and tensors comprising the tGSVD described above are unique up to a phase factor of ±1 in each element of the core tensors, except in the case of degenerate subspaces, defined by subsets of equal angular distances (i.e. relative significance) in the mGSVD calculation.

Corollary 1 (Reduction to mGSVD).

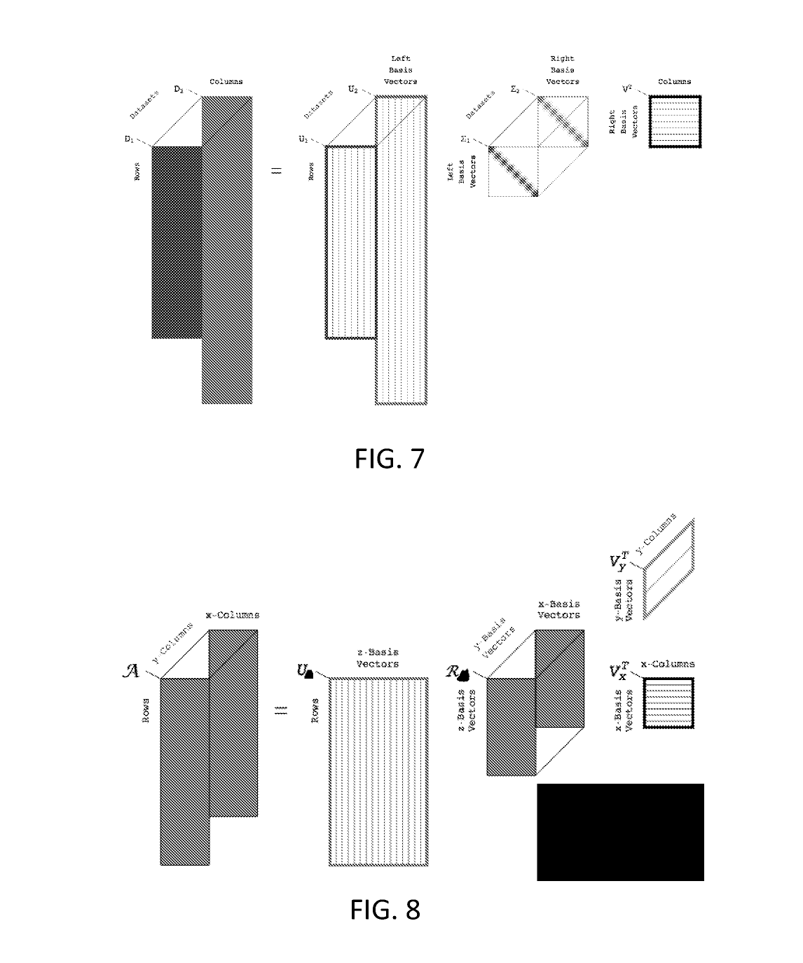

Let A and B be matrices of full column rank with I_{1,A} and I_{1,B} number of rows, respectively, and both with I₂ columns. Also let min {I_{1,A}, I_{1,B}}>I₂. The tGSVD of A and B is equivalent to the mGSVD of A and B, as shown in FIG. 7.

Theorem 1.

The mGSVD of two matrices, A and B, reduces to the SVD of A if B is of the form,

$$B = [ \begin{matrix}{I}_{n} \\ 0\end{matrix} ]$$

where Iₙ is the n×n identity matrix. Theorem 1 shows that the mGSVD, performed on the unfoldings of [symbol] and [symbol] on every axis, becomes the SVD of A₍ₙ₎ on each axis, which is exactly how the HOSVD of [symbol] is constructed.

Corollary 2.

Let [symbol] and [symbol] be tensors with N dimensions of size

[symbol]

>I₂=I₃= . . . =I_{N}

[symbol]

>I₂=I₃= . . . =I_{N},

respectively. Also let [symbol] have ones on the diagonal, i.e. when all indices are equal, and zeros everywhere else. Then, the tGSVD of [symbol] and [symbol] is equivalent to the HOSVD of [symbol] , as shown in FIG. 8.

Theorem 2.

The relative significance in the tGSVD defined as the ratio of corresponding entries in [symbol] and [symbol] , i.e. [symbol] _{, i}_{₁}_{, i}_{₂} _{. . . i}_{₃}/ [symbol] _{, i}_{₁}ᵢ_{₂} _{. . . i}_{₃}, depends only on the first index, i₁, and is identical to the relative significance of the mGSVD of [symbol] and [symbol] unfolded to preserve the first axis (i.e., the first unfolding of the data tensors, [symbol] ₍₁₎ and [symbol] ₍₁₎ by preserving the row axis).

Therefore, the tGSVD exists and is unique up to sign in the core tensor. The tGSVD reduces to the mGSVD when second order tensors (i.e., matrices) are given as inputs. The tGSVD reduces to the Higher Order SVD when one of the input tensors has ones on the diagonal (i.e., when all indices are equal) and zeros everywhere else.

Ideally, the matrix HO GSVD's left basis vectors Uᵢ would be column-wise orthogonal also outside of the common subspace of the N matrices. An iterative matrix block HO GSVD can be defined. First, the common subspace of all N matrices Dᵢ is used to separate each of the matrices Uᵢ into a column-wise orthogonal block ∈ [symbol] ^{m}^{ᵢ}^{×k} and the remaining block. Next, the HO GSVD of the blocks ∈ [symbol] ^{m}^{ᵢ}^{×(n-k)} of a subset of, e.g., N−1 matrices UᵢΣᵢ (that correspond to the remaining blocks in Uᵢ) is used to identify the subspace common to the N−1 but not all N matrices Dᵢ. The column-wise orthogonal blocks that correspond to the N−1 (but not to the N) common subspace are used to rewrite the corresponding blocks of Uᵢ that previously were not necessarily orthogonal. This step is repeated until all matrices Uᵢ are completely column-wise orthogonal. Thus, the matrix HO GSVD is a special case of this iterative matrix block HO GSVD.

### Example 4

To compare two datasets that are each of higher order than a matrix (e.g. order 3 tensors), the tGSVD simultaneously separates the paired datasets into paired weighted sums of subtensors, formed by the outer product of a single pattern of variation across each dimension, as shown above. The significance of the subtensor [symbol] (i₁, i₂, . . . , i_{N}) for [symbol] ∈{ [symbol] , [symbol] }, in the dataset [symbol] , in terms of the overall information that it captures in this dataset, is proportional to the weight of the i₁, i₂, . . . , i_{N} entry of [symbol] , i.e.,

$${ρ}_{τ , {i}_{1} {i}_{2} … {i}_{N}} = \frac{{r}_{τ , {i}_{1} {i}_{2} … {i}_{N}}^{2}}{{‖ {ℛ}_{τ} ‖}^{2}} .$$

The “Shannon entropy” of each dataset,

$$0 \leq d = \frac{- 1}{2 ( {I}_{2} {I}_{3} … {I}_{N} )} {\sum }_{{i}_{1} = 1}^{{I}_{2} {I}_{3} … {I}_{N}} {\sum }_{{i}_{2} = 1}^{{I}_{2}} … {\sum }_{{i}_{N} = 1}^{{I}_{N}} {p}_{τ , {i}_{1} {i}_{2} … {i}_{N}} log {p}_{τ , {i}_{1} {i}_{2} … {i}_{N}} \leq 1$$

measures the complexity of the data from the distribution of the overall information among the different subtensors. An entropy of zero corresponds to an ordered and redundant dataset in which all the information is captured by a single subtensor. An entropy of one corresponds to a disordered and random dataset in which all subtensors are of equal significance. The significance of the subtensor [symbol] (i₁, i₂, . . . , i_{N}) in [symbol] relative to the significance of [symbol] (i₁, i₂, . . . , i_{N}) in [symbol] is defined in terms of an “angular distance,”θᵢ_{₁}_{, i}_{₂}_{, . . . , i}_{_{N}}, that is proportional to the ratio of the corresponding weights,

−π/4≤θᵢ_{₁}_{,i}_{₂}_{, . . . ,i}_{_{N}}=arctan( [symbol] _{,i}_{₁}_{,i}_{₂}_{, . . . ,i}_{₃}/ [symbol] _{,i}_{₁}_{,i}_{₂}_{, . . . ,i}_{₃})−π/4≤−π/4.

### Example 5

An angular distance of −π/4 indicates a subtensor that is exclusive to either dataset [symbol] or [symbol] , respectively, whereas an angular distance of zero indicates a subtensor that is common to both datasets [symbol] and [symbol] . Note that the corresponding subtensors [symbol] (i₁, i₂, . . . , i_{N}) and [symbol] (i₁, i₂, . . . , i_{N}), are constructed as an outer product of identical columns from each of the matrices Vn and corresponding non-identical columns of [symbol] and [symbol] . Theorem 2 proves that the relative significance depends on the row index only. Therefore, only columns of [symbol] and [symbol] contribute to the relative significance whereas columns of Vn contribute to significance within each dataset independently.

The subject technology provides frameworks that can simultaneously compare and contrast two datasets arranged in large-scale tensors of the same column dimensions but with different row dimensions in order to find the similarities and dissimilarities among them. The subject technology may be applied in fields such as medicine, where the number of high-dimensional datasets, recording multiple aspects of a disease across the same set of patients, is increasing, such as in The Cancer Genome Atlas (TCGA).

For example, despite recent large-scale profiling efforts, the best prognostic predictor of glioblastoma multiforme (GBM) has been the patient's age at diagnosis. A global pattern of tumor-exclusive co-occurring copy-number alterations (CNAs) is correlated, possibly coordinated with GBM patients' survival and response to chemotherapy. The pattern was revealed by generalized singular value decomposition (GSVD) comparison of patient-matched but probe-independent GBM and normal array CGH datasets from TCGA (FIG. 9).

According to some embodiments of the subject technology, the GSVD, formulated as a framework for comparatively modeling two composite datasets, removes from the pattern copy-number variations (CNVs) that occur in the normal human genome (e.g., female-specific X chromosome amplification) and experimental variations (e.g., in tissue batch, genomic center, hybridization date and scanner), without a-priori knowledge of these variations. Second, the pattern includes most known GBM-associated changes in chromosome numbers and focal CNAs, as well as several previously unreported CNAs in >3% of the patients. These include the biochemically putative drug target, cell cycle-regulated serine/threonine kinase-encoding TLK2, the cyclin E1-encoding CCNE1, and the Rb-binding histone demethylase-encoding KDM5A. Third, the pattern provides a better prognostic predictor than the chromosome numbers or any one focal CNA that it identifies, suggesting that the GBM survival phenotype is an outcome of its global genotype. The pattern is independent of age, and combined with age, makes a better predictor than age alone.

Similarly, the best predictor of the ovarian serous cystadenocarcinoma (OV) remains the tumor's stage, an assessment—numbering I to IV—of the spread of the cancer. To identify CNAs that might predict OV patients' survival, patient- and platform-matched OV and normal copy-number profiles can be comparatively modeled by using a novel tensor GSVD. This tensor GSVD enables the simultaneous decomposition of two datasets arranged in higher-order tensors, whereas the matrix GSVD is limited to two second-order tensors, i.e., matrices. The additional dimension allows separation of platform bias.

A tensor GSVD can be defined for two large-scale tensors with different row dimensions and the same column dimensions. The tensor GSVD provides a framework for comparative modeling in personalized medicine, where the mathematical variables represent biomedical reality. Just as the matrix GSVD enabled the discovery of CNAs correlated with GBM survival, the tensor GSVD enables a comparison of two, higher dimensional datasets leading to the discovery of CNAs that are correlated with OV prognosis. This mathematical modeling makes it possible to similarly use recent high-throughput biotechnologies in the personalized prognosis and treatment of OV and other cancers.

The pattern of particular biomedical interest is the most significant in the tumor dataset (i.e. the one that captures the largest fraction of information), is independent of platform, and is exclusive to the tumor dataset. To build this subtensor, the most significant pattern in the tumor data is used for V_{x,b}, the most platform-independent pattern for V_{y,c}, and the most tumor exclusive pattern, determined by relative significance, is used for U_{B,a}.

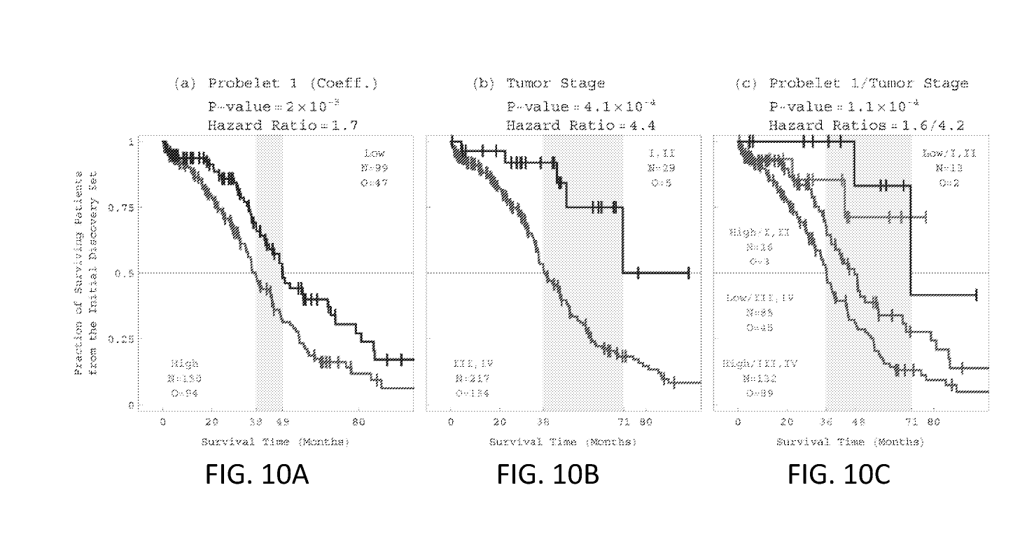

As shown in FIGS. 10A-C, an exemplary embodiment of the tensor GSVD with TCGA data can be illustrated by comparing normal and OV tumor genomic profiles from the same set of patients, each measured twice by the same two profiling platforms. The tensor GSVD has uncovered several tumor-exclusive chromosome arm-wide patterns of CNAs that are consistent across both profiling platforms and are significantly correlated with the patients' survival. This indicates several, previously unrecognized, subtypes of OV. The prognostic contributions of these patterns are comparable to and independent of the tumor's stage (FIGS. 10A-C). Tensor GSVD classification of the OV profiles of an independent set of patients validates the prognostic contribution of these patterns.

### Example 6

According to some embodiments, methods of the subject technology can be implemented in the field of epidemiology. For example, data relating to infection rates can be tabulated in tensors. Each tensor can represent or contain values for infection rate data for a given region (e.g., continent, country, state, county, city, district, etc.). The shared x-axis can represent or contain values for time. The shared y-axis can represent or contain values for infectious diseases. The z-axis can represent or contain values for sub-regions (e.g., state, county, city, district, etc.) within the corresponding region represented by the tensor. The tensor GSVD and/or HO GSVD can be performed to determine similarities and dissimilarities between two regions or among three or more regions with respect to infection rates of different diseases across time.

### Example 7

According to some embodiments, methods of the subject technology can be implemented in the field of agriculture. For example, data relating to crop yields can be tabulated in tensors. Each tensor can represent or contain values for crop yield data for a given crop (e.g., corn, rice, wheat, etc.). The shared x-axis can represent or contain values for time. The shared y-axis (or multiple y-axes) can represent or contain values for geocoordinates. The z-axis (or multiple z-axes) can represent or contain values for different types of a given crop (e.g., different types of corn, different types of rice, different types of wheat, etc.). The tensor GSVD and/or HO GSVD can be performed to determine similarities and dissimilarities between the yields of two crops (or among more than two) across time and geocoordinates.

### Example 8

According to some embodiments, methods of the subject technology can be implemented in the field of ecology. For example, data relating to abundance levels can be tabulated in tensors. Each tensor can represent or contain values for abundance level data for a given disease vector (e.g., virus, fungi, pollen, etc.). The shared x-axis can represent or contain values for time. The shared y-axis (or multiple y-axes) can represent or contain values for geocoordinates. The z-axis (or multiple z-axes) can represent or contain values for different types of a given disease vector (e.g., different types of virus, different types of fungi, different types of pollen, etc.). The tensor GSVD and/or HO GSVD can be performed to similarities and dissimilarities between the abundance levels of two disease vectors (or among more than two) across time and geocoordinate.

### Example 9

According to some embodiments, methods of the subject technology can be implemented in the field of political science. For example, data relating to poll numbers can be tabulated in tensors. Each tensor can represent or contain values for polling data for a given voting territory (e.g., state, county, district, etc.). The shared x-axis can represent or contain values for time. The shared y-axis (or multiple y-axes) can represent or contain values for candidates and/or issues. Additional or alternative possible shared axes can include demographic factors (e.g., age, income, occupation, marital status, number of children, party membership, etc.). The z-axis (or multiple z-axes) can represent or contain values for sub-territories (e.g., precincts, etc.) within the corresponding voting territory represented by the tensor. The tensor GSVD and/or HO GSVD can be performed to determine similarities and dissimilarities between public opinion on candidates or issues in two states (or among more than two) across time.

### Example 10

According to some embodiments, methods of the subject technology can be implemented in the field of macroeconomics. For example, data relating to employment rates can be tabulated in tensors. One or more tensors can represent or contain values for employment data such as employment rate, government spending in dollars, levels of macroeconomic factors (e.g., tax rates, interest rates, etc.). The shared x-axis can represent or contain values for time. The shared y-axis (or multiple y-axes) can represent or contain values for regions (e.g., continent, country, state, county, city, district, etc.). The z-axis (or multiple z-axes) can represent or contain values for different areas of government spending and/or different types of macroeconomic factors (e.g., types of taxes, types of interest rates, etc.). The tensor GSVD and/or HO GSVD can be performed to determine similarities and dissimilarities between the two macroeconomic factors of employment and government spending (or among more than two factors, including, e.g., taxes, or interest rates) across time and cities.

### Example 11

According to some embodiments, methods of the subject technology can be implemented in the field of finance. For example, data relating to prices can be tabulated in tensors. Each tensor can represent or contain values for pricing data for a given asset or assets (e.g., stock prices, commodity prices, etc.) and/or pricing factors (e.g., housing prices). The shared x-axis can represent or contain values for time. The shared y-axis (or multiple y-axes) can represent or contain values for region(s). The z-axis (or multiple z-axes) can represent or contain values for different ones of the asset or assets (e.g., different stocks, different commodities, different pricing factors, etc.). The tensor GSVD and/or HO GSVD can be performed to determine similarities and dissimilarities between the two finance factors of stocks and commodities (or among more than two factors, including, e.g., housing prices) across time and regions.

### Example 12

According to some embodiments, methods of the subject technology can be implemented in the field of sports. For example, data relating to sports statistics (e.g., offensive statistics, on-base percentage, defensive statistics, earned run average, etc.) can be tabulated in tensors for one or more teams, players, or other participants. The statistics can relate to performance, results, training, and/or environmental factors. Each tensor can represent or contain values for statistical data for a given team, player, or other participant. The shared x-axis can represent or contain values for a span of time or group of events (e.g., season, game, inning, quarter, period, etc.). The shared y-axis (or multiple y-axes) can represent or contain values for game information, such as opposing team, location, opposing players, weather, time, duration, etc. The z-axis (or multiple z-axes) can represent or contain values for players or other participants corresponding to particular teams, for example. The tensor GSVD and/or HO GSVD can be performed to determine similarities and dissimilarities between the two teams (or among more than two teams) across season and games in season.

### Example 13

According to some embodiments, methods of the subject technology can be implemented in the field of traffic analysis. For example, data relating to traffic can be tabulated in tensors. Each tensor can represent a location (e.g., intersection, length of road, etc.) and contain values for individual experience (e.g., time that a car spends in a traffic intersection on each occasion, or mean speed of the car on a road on each occasion, etc.). The shared x-axis can represent or contain values for time (e.g., time of day, etc.). The shared y-axis (or multiple y-axes) can also represent or contain values for time (e.g., day of the week, etc.). The z-axis (or multiple z-axes) can represent or contain values for vehicles that travel through the corresponding location represented by the tensors. The tensor GSVD and/or HO GSVD can be performed to determine similarities and dissimilarities between the two traffic intersections, or roads (or among more than two intersections, or roads) across time of day, and day of the week, in terms of time spent, or mean speed driven.

### Example 14

According to some embodiments, methods of the subject technology can be implemented in the field of social media applications. For example, data relating to social media activity can be tabulated in tensors. Each tensor can represent or contain values for a number of posts (e.g., tweets, notifications, submissions, uploads, etc.) or individuals posting for a given identifier (e.g., hashtag, etc.). The shared x-axis can represent or contain values for time. The shared y-axis (or multiple y-axes) can represent or contain values for regions (e.g., continent, country, state, county, city, district, etc.). Additional or alternate possible shared axes include demographic factors (e.g., age, sex, income, occupation, relationship status, number of children, religious affiliation, political party membership, etc.). The z-axis (or multiple z-axes) can represent or contain values for people or number of people posting with a given identifier. The tensor GSVD and/or HO GSVD can be performed to determine similarities and dissimilarities between the levels of discussion of two hashtags (or among more than two) over time and in different regions (e.g., cities).

### Example 15

According to some embodiments, methods of the subject technology can be implemented in the field of climate and environment. For example, data relating to climate can be tabulated in tensors. Each tensor can represent or contain values for climate data for a given factor (e.g., atmosphere characteristics, infrared clouds, chemistry, ozone, aerosols, outgoing long wave energy, ocean characteristics, dissolved oxygen at different depths, land characteristics, vegetation, cryosphere characteristics, snow and ice cover, and climate, observations, simulations, factors created by humans, chemical characteristics, light pollution characteristics, geophysical measurements, satellite observations, data from the National Oceanic and Atmospheric Administration, biological measurements, abundance levels, genomic sequences of living organisms, etc.). The shared x-axis can represent or contain values for location (e.g., latitude, etc.). The shared y-axis (or multiple y-axes) can represent or contain values for location (e.g., longitude, etc.). Additional possible shared axes can include geophysical factors (e.g., elevation, day in the year, etc.). The tensor GSVD and/or HO GSVD can be performed to determine similarities and dissimilarities between the variations of two climate and environmental factors (or among more than two) across latitude and longitude (and possibly also, e.g., elevation, and day in the year).

### Example 16

According to some embodiments, methods of the subject technology can be implemented in the field of recommendation systems. For example, data relating to recommendations can be tabulated in tensors. Each tensor can represent or contain values for recommendation data for a given user (e.g., user identity, type of media, experience ratings, etc.). The shared x-axis (or multiple x-axes) and the shared y-axis (or multiple y-axes) can represent or contain values for demographic factors (e.g., income level, state, or city). The z-axis (or multiple z-axes) can represent or contain values for types of examples of media or other consumer products and services (e.g., movies, books, music, dining, vacation locations, etc.). The tensor GSVD and/or HO GSVD can be performed to determine similarities and dissimilarities between user, or experience ratings of movies and books (or among more than two consumer products, including, e.g., vacation sites) across consumer demographics (e.g., income level, location, state, city, etc.). The tensor GSVD can also be used to help individuals make life decisions such as college, field of study, where to live, etc., provided that some sort of quantified information (e.g., subject's satisfaction on a scale of 1 to 10) is available. Shared axes could include demographic data, grades, test scores, membership in various organizations, etc. This data could be cross-correlated with other fields (e.g., social media, politics) that have similar demographic data as shared axes.

### Example 17

According to some embodiments, methods of the subject technology can be implemented in the field of fitness management. For example, data relating to fitness (e.g., frequencies or levels of one type of exercise, frequencies or amounts of any one food, SNP profiles, measured, e.g., by DNA microarrays, etc.) can be tabulated in tensors. Each tensor can represent or contain values for fitness data for a given user. The shared x-axis can represent or contain values for vital signs (e.g., blood pressure, heart rate, etc.). Additional possible shared axes can include additional fitness factors (e.g., additional vital signs, weight, cholesterol levels), life style indicators (e.g., occupation), and family history. Tensors can correspond to exercise data, nutrition data, and/or any one of additional possible effectors of fitness (e.g., genetics as measured by, e.g., single-nucleotide polymorphism, i.e., SNP, profile, etc.) The z-axis (or multiple z-axes) can represent or contain values for different types of exercises, different types of foods, different probes of a SNP profile. The tensor GSVD and/or HO GSVD can be performed to determine similarities and dissimilarities between the two fitness effectors of exercise and nutrition (or among more than two fitness effectors, including, e.g., genetics) and their correlations with two or more fitness factors, e.g., vital signs, life style indicators, and family history.

### Example 18

According to some embodiments, methods of the subject technology can be implemented in the field of marketing and advertising. For example, data relating to numbers of purchases can be tabulated in tensors. Each tensor can represent or contain values for purchase data for a given source of goods and/or services (e.g., store, chain of stores, website, etc.). The shared x-axis can represent or contain values for a first demographic factor (e.g., income level, etc.). The shared y-axis (or multiple y-axes) can represent or contain values for a second demographic factor (e.g., state or city, etc.). The z-axis (or multiple z-axes) can represent or contain values for different items from one or more stores (e.g., different items from store 1, or chain 1, different items from store 2, or chain 2, different items from store 3, or chain 3, etc.). The tensor GSVD and/or HO GSVD can be performed to determine similarities and dissimilarities between purchases in two stores or chains (or among more than two stores) across consumer demographics, e.g., income level, and state or city. This could also be used to inform, e.g., targeted advertising.

### Example 19

According to some embodiments, methods of the subject technology can be implemented in the field of astrophysics. For example, data relating to intensities can be tabulated in tensors. Each tensor can represent or contain values for data from a given telescope and/or operating parameter (e.g., frequency, etc.). The shared x-axis can represent or contain values for first celestial coordinates. The shared y-axis (or multiple y-axes) can represent or contain values for second celestial coordinates. The z-axis (or multiple z-axes) can represent or contain values for time points measured by different telescopes. The tensor GSVD and/or HO GSVD can be performed to determine similarities and dissimilarities between sky surveys of two telescopes (or among more than two telescopes) at the same or different frequencies across celestial coordinates. Dissimilar variations might correspond to experimental variation between the two (or among the more than two) telescopes. Similarities might correspond to different recordings of the same astrophysical event by the two, or more telescopes.

### Example 20

According to some embodiments, methods of the subject technology can be implemented in the field of voice and speech recognition. For example, data relating to intensities can be tabulated in tensors. Each tensor can represent or contain values for data for a given user. The shared x-axis can represent or contain values for a first speech characteristic (e.g., phonemes, etc.). The shared y-axis (or multiple y-axes) can represent or contain values for a second speech characteristic (e.g., notes, etc.). The z-axis (or multiple z-axes) can represent or contain values for time points in a recording of a corresponding user. The tensor GSVD and/or HO GSVD can be performed to determine similarities and dissimilarities between two speakers or singers (or among more than two) across commonly defined speech characteristics. This might identify the speech characteristics signature of each individual person, and be used in voice recognition.

### Example 21

According to some embodiments, methods of the subject technology can be implemented in the field of natural language processing and machine translation. For example, data relating to term frequency-inverse document frequencies (TF-IDFs) can be tabulated in tensors. Each tensor can represent or contain values for data for a given language. The shared x-axis can represent or contain values for books or other literary works. The shared y-axis (or multiple y-axes) can represent or contain values for chapters and/or verses. The z-axis (or multiple z-axes) can represent or contain values for N-grams (e.g., phonemes, syllables, letters, words, etc.) with respect to the corresponding language represented by the tensor. The tensor GSVD and/or HO GSVD can be performed to determine similarities and dissimilarities between two languages (or among more than two languages) in TF-IDFs of different n-grams across books and chapters in books.

### Example 22

According to some embodiments, methods of the subject technology can be implemented in the field of market demand and manufacturing. For example, data relating to market activity can be tabulated in tensors. Each tensor can represent or contain values for market data for a given indicator (e.g., number of items sold, value of items sold, employment rate, weather indicator, time, etc.). The shared x-axis can represent or contain values for location. The shared y-axis (or multiple y-axes) can represent or contain values for time (e.g., day in the year). The z-axis (or multiple z-axes) can represent or contain values for availability of an item (e.g., measures in time span, etc.). The tensor GSVD and/or HO GSVD can be performed to determine similarities and dissimilarities between sales and an effector of sales, e.g., an economic indicator (or among sales, more than one effector, including, e.g., weather) and their correlations with location and day in the year. This could be used to predict market demand, and tailor manufacturing.

### Example 23

According to some embodiments, methods of the subject technology can be implemented in the field of education and personal development. For example, data relating to student characteristics can be tabulated in tensors. Each tensor can represent or contain values for student data (e.g., books read, etc.) for a given characteristic (e.g., GPA, school attended, etc.). The shared x-axis (or multiple x-axes) and the shared y-axis (or multiple y-axes) can represent or contain values for demographic factors (e.g., income level of parents, state or city of high school, etc.). The z-axis (or multiple z-axes) can represent or contain values for books read (e.g., list of books read by at least one student with GPA 4.0, list of books read by at least one student with GPA 3.0, list of books read by at least one student with GPA 2.0, etc.). The tensor GSVD and/or HO GSVD can be performed to determine similarities and dissimilarities between students with GPA 4.0 and 3.0 (or among more than two groups of students, including, e.g., those with GPA 2.0) across demographic factors, and in terms of books read or unread. This could be used to identify the reading habits that are exclusive to students with high, 4.0 GPA at University X.

### Systems

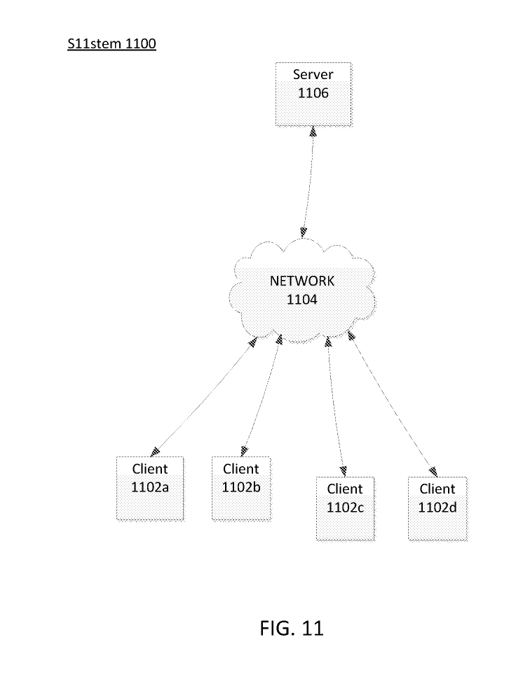

FIG. 11 is a simplified diagram of a system 1100, in accordance with various embodiments of the subject technology. The system 1100 may include one or more remote client devices 1102 (e.g., client devices 1102a, 1102b, 1102c, 1102d, and 1102e) in communication with one or more server computing devices 1106 (e.g., servers 1106a and 1106b) via network 1104. In some embodiments, a client device 1102 is configured to run one or more applications based on communications with a server 1106 over a network 1104. In some embodiments, a server 1106 is configured to run one or more applications based on communications with a client device 1102 over the network 1104. In some embodiments, a server 1106 is configured to run one or more applications that may be accessed and controlled at a client device 1102. For example, a user at a client device 1102 may use a web browser to access and control an application running on a server 1106 over the network 1104. In some embodiments, a server 1106 is configured to allow remote sessions (e.g., remote desktop sessions) wherein users can access applications and files on a server 1106 by logging onto a server 1106 from a client device 1102. Such a connection may be established using any of several well-known techniques such as the Remote Desktop Protocol (RDP) on a Windows-based server.

By way of illustration and not limitation, in some embodiments, stated from a perspective of a server side (treating a server as a local device and treating a client device as a remote device), a server application is executed (or runs) at a server 1106. While a remote client device 1102 may receive and display a view of the server application on a display local to the remote client device 1102, the remote client device 1102 does not execute (or run) the server application at the remote client device 1102. Stated in another way from a perspective of the client side (treating a server as remote device and treating a client device as a local device), a remote application is executed (or runs) at a remote server 1106.

By way of illustration and not limitation, in some embodiments, a client device 1102 can represent a desktop computer, a mobile phone, a laptop computer, a netbook computer, a tablet, a thin client device, a personal digital assistant (PDA), a portable computing device, and/or a suitable device with a processor. In one example, a client device 1102 is a smartphone (e.g., iPhone, Android phone, Blackberry, etc.). In certain configurations, a client device 1102 can represent an audio player, a game console, a camera, a camcorder, a Global Positioning System (GPS) receiver, a television set top box an audio device, a video device, a multimedia device, and/or a device capable of supporting a connection to a remote server. In some embodiments, a client device 1102 can be mobile. In some embodiments, a client device 1102 can be stationary. According to certain embodiments, a client device 1102 may be a device having at least a processor and memory, where the total amount of memory of the client device 1102 could be less than the total amount of memory in a server 1106. In some embodiments, a client device 1102 does not have a hard disk. In some embodiments, a client device 1102 has a display smaller than a display supported by a server 1106. In some aspects, a client device 1102 may include one or more client devices.

In some embodiments, a server 1106 may represent a computer, a laptop computer, a computing device, a virtual machine (e.g., VMware® Virtual Machine), a desktop session (e.g., Microsoft Terminal Server), a published application (e.g., Microsoft Terminal Server), and/or a suitable device with a processor. In some embodiments, a server 1106 can be stationary. In some embodiments, a server 1106 can be mobile. In certain configurations, a server 1106 may be any device that can represent a client device. In some embodiments, a server 1106 may include one or more servers.

In some embodiments, a first device is remote to a second device when the first device is not directly connected to the second device. In some embodiments, a first remote device may be connected to a second device over a communication network such as a Local Area Network (LAN), a Wide Area Network (WAN), and/or other network.

When a client device 1102 and a server 1106 are remote with respect to each other, a client device 1102 may connect to a server 1106 over the network 1104, for example, via a modem connection, a LAN connection including the Ethernet or a broadband WAN connection including DSL, Cable, T1, T3, Fiber Optics, Wi-Fi, and/or a mobile network connection including GSM, GPRS, 3G, 4G, 4G LTE, WiMax or other network connection. Network 1104 can be a LAN network, a WAN network, a wireless network, the Internet, an intranet, and/or other network. The network 1104 may include one or more routers for routing data between client devices and/or servers. A remote device (e.g., client device, server) on a network may be addressed by a corresponding network address, such as, but not limited to, an Internet protocol (IP) address, an Internet name, a Windows Internet name service (WINS) name, a domain name, and/or other system name. These illustrate some examples as to how one device may be remote to another device, but the subject technology is not limited to these examples.

According to certain embodiments of the subject technology, the terms “server” and “remote server” are generally used synonymously in relation to a client device, and the word “remote” may indicate that a server is in communication with other device(s), for example, over a network connection(s).

According to certain embodiments of the subject technology, the terms “client device” and “remote client device” are generally used synonymously in relation to a server, and the word “remote” may indicate that a client device is in communication with a server(s), for example, over a network connection(s).

In some embodiments, a “client device” may be sometimes referred to as a client or vice versa. Similarly, a “server” may be sometimes referred to as a server device or server computer or like terms.

In some embodiments, the terms “local” and “remote” are relative terms, and a client device may be referred to as a local client device or a remote client device, depending on whether a client device is described from a client side or from a server side, respectively. Similarly, a server may be referred to as a local server or a remote server, depending on whether a server is described from a server side or from a client side, respectively. Furthermore, an application running on a server may be referred to as a local application, if described from a server side, and may be referred to as a remote application, if described from a client side.

In some embodiments, devices placed on a client side (e.g., devices connected directly to a client device(s) or to one another using wires or wirelessly) may be referred to as local devices with respect to a client device and remote devices with respect to a server. Similarly, devices placed on a server side (e.g., devices connected directly to a server(s) or to one another using wires or wirelessly) may be referred to as local devices with respect to a server and remote devices with respect to a client device.

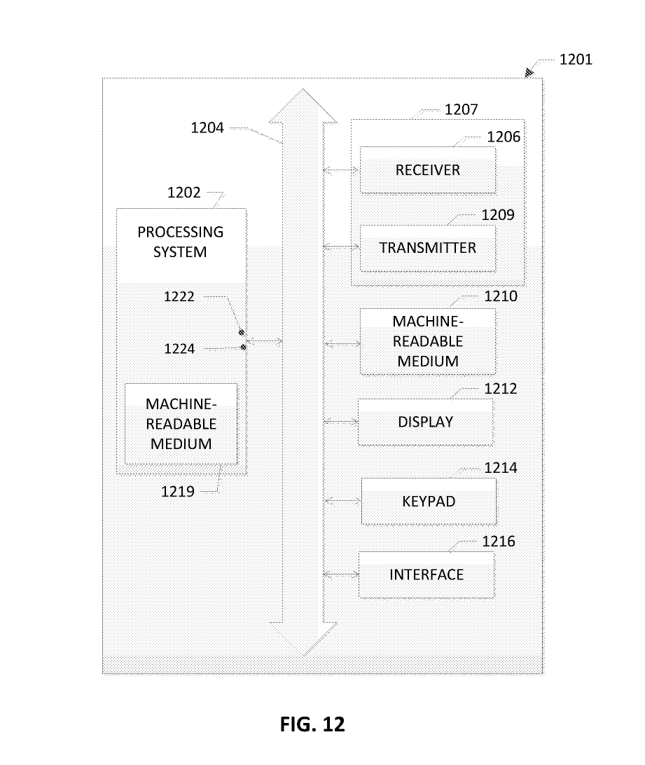

FIG. 12 is a block diagram illustrating an exemplary computer system 1200 with which a client device 1102 and/or a server 1106 of FIG. 11 can be implemented. In certain embodiments, the computer system 1200 may be implemented using hardware or a combination of software and hardware, either in a dedicated server, or integrated into another entity, or distributed across multiple entities.

The computer system 1200 (e.g., client 1102 and servers 1106) includes a bus 1208 or other communication mechanism for communicating information, and a processor 1202 coupled with the bus 1208 for processing information. By way of example, the computer system 1200 may be implemented with one or more processors 1202. The processor 1202 may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, and/or any other suitable entity that can perform calculations or other manipulations of information.

The computer system 1200 can include, in addition to hardware, code that creates an execution environment for the computer program in question, e.g., code that constitutes processor firmware, a protocol stack, a database management system, an operating system, or a combination of one or more of them stored in an included memory 1204, such as a Random Access Memory (RAM), a flash memory, a Read Only Memory (ROM), a Programmable Read-Only Memory (PROM), an Erasable PROM (EPROM), registers, a hard disk, a removable disk, a CD-ROM, a DVD, and/or any other suitable storage device, coupled to the bus 1208 for storing information and instructions to be executed by the processor 1202. The processor 1202 and the memory 1204 can be supplemented by, or incorporated in, special purpose logic circuitry.

The instructions may be stored in the memory 1204 and implemented in one or more computer program products, i.e., one or more modules of computer program instructions encoded on a computer readable medium for execution by, or to control the operation of, the computer system 1200, and according to any method well known to those of skill in the art, including, but not limited to, computer languages such as data-oriented languages (e.g., SQL, dBase), system languages (e.g., C, Objective-C, C++, Assembly), architectural languages (e.g., Java, .NET), and/or application languages (e.g., PHP, Ruby, Perl, Python). Instructions may also be implemented in computer languages such as array languages, aspect-oriented languages, assembly languages, authoring languages, command line interface languages, compiled languages, concurrent languages, curly-bracket languages, dataflow languages, data-structured languages, declarative languages, esoteric languages, extension languages, fourth-generation languages, functional languages, interactive mode languages, interpreted languages, iterative languages, list-based languages, little languages, logic-based languages, machine languages, macro languages, metaprogramming languages, multiparadigm languages, numerical analysis, non-English-based languages, object-oriented class-based languages, object-oriented prototype-based languages, off-side rule languages, procedural languages, reflective languages, rule-based languages, scripting languages, stack-based languages, synchronous languages, syntax handling languages, visual languages, wirth languages, and/or xml-based languages. The memory 1204 may also be used for storing temporary variable or other intermediate information during execution of instructions to be executed by the processor 1202.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network. The processes and logic flows described in this specification can be performed by one or more programmable processors executing one or more computer programs to perform functions by operating on input data and generating output.

The computer system 1200 further includes a data storage device 1206 such as a magnetic disk or optical disk, coupled to the bus 1208 for storing information and instructions. The computer system 1200 may be coupled via an input/output module 1210 to various devices (e.g., devices 1214 and 1216). The input/output module 1210 can be any input/output module. Exemplary input/output modules 1210 include data ports (e.g., USB ports), audio ports, and/or video ports. In some embodiments, the input/output module 1210 includes a communications module. Exemplary communications modules include networking interface cards, such as Ethernet cards, modems, and routers. In certain aspects, the input/output module 1210 is configured to connect to a plurality of devices, such as an input device 1214 and/or an output device 1216. Exemplary input devices 1214 include a keyboard and/or a pointing device (e.g., a mouse or a trackball) by which a user can provide input to the computer system 1200. Other kinds of input devices 1214 can be used to provide for interaction with a user as well, such as a tactile input device, visual input device, audio input device, and/or brain-computer interface device. For example, feedback provided to the user can be any form of sensory feedback (e.g., visual feedback, auditory feedback, and/or tactile feedback), and input from the user can be received in any form, including acoustic, speech, tactile, and/or brain wave input. Exemplary output devices 1216 include display devices, such as a cathode ray tube (CRT) or liquid crystal display (LCD) monitor, for displaying information to the user.

According to certain embodiments, a client device 1102 and/or server 1106 can be implemented using the computer system 1200 in response to the processor 1202 executing one or more sequences of one or more instructions contained in the memory 1204. Such instructions may be read into the memory 1204 from another machine-readable medium, such as the data storage device 1206. Execution of the sequences of instructions contained in the memory 1204 causes the processor 1202 to perform the process steps described herein. One or more processors in a multi-processing arrangement may also be employed to execute the sequences of instructions contained in the memory 1204. In some embodiments, hard-wired circuitry may be used in place of or in combination with software instructions to implement various aspects of the present disclosure. Thus, aspects of the present disclosure are not limited to any specific combination of hardware circuitry and software.

Various aspects of the subject matter described in this specification can be implemented in a computing system that includes a back end component (e.g., a data server), or that includes a middleware component (e.g., an application server), or that includes a front end component (e.g., a client computer having a graphical user interface and/or a Web browser through which a user can interact with an implementation of the subject matter described in this specification), or any combination of one or more such back end, middleware, or front end components. The components of the system 1200 can be interconnected by any form or medium of digital data communication (e.g., a communication network). Examples of communication networks include a local area network and a wide area network.

The term “machine-readable storage medium” or “computer readable medium” as used herein refers to any medium or media that participates in providing instructions to the processor 1202 for execution. Such a medium may take many forms, including, but not limited to, non-volatile media, volatile media, and transmission media. Non-volatile media include, for example, optical or magnetic disks, such as the data storage device 1206. Volatile media include dynamic memory, such as the memory 1204. Transmission media include coaxial cables, copper wire, and fiber optics, including the wires that comprise the bus 1208. Common forms of machine-readable media include, for example, floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH EPROM, any other memory chip or cartridge, or any other medium from which a computer can read. The machine-readable storage medium can be a machine-readable storage device, a machine-readable storage substrate, a memory device, a composition of matter effecting a machine-readable propagated signal, or a combination of one or more of them.

As used herein, a “processor” can include one or more processors, and a “module” can include one or more modules.

In an aspect of the subject technology, a machine-readable medium is a computer-readable medium encoded or stored with instructions and is a computing element, which defines structural and functional relationships between the instructions and the rest of the system, which permit the instructions' functionality to be realized. Instructions may be executable, for example, by a system or by a processor of the system. Instructions can be, for example, a computer program including code. A machine-readable medium may comprise one or more media.

As used herein, the word “module” refers to logic embodied in hardware or firmware, or to a collection of software instructions, possibly having entry and exit points, written in a programming language, such as, for example C++. Two or more modules may be embodied in a single piece of hardware, firmware or software. A software module may be compiled and linked into an executable program, installed in a dynamic link library, or may be written in an interpretive language such as BASIC. It will be appreciated that software modules may be callable from other modules or from themselves, and/or may be invoked in response to detected events or interrupts. Software instructions may be embedded in firmware, such as an EPROM or EEPROM. It will be further appreciated that hardware modules may be comprised of connected logic units, such as gates and flip-flops, and/or may be comprised of programmable units, such as programmable gate arrays or processors. The modules described herein are preferably implemented as software modules, but may be represented in hardware or firmware.

It is contemplated that the modules may be integrated into a fewer number of modules. One module may also be separated into multiple modules. The described modules may be implemented as hardware, software, firmware or any combination thereof. Additionally, the described modules may reside at different locations connected through a wired or wireless network, or the Internet.

In general, it will be appreciated that the processors can include, by way of example, computers, program logic, or other substrate configurations representing data and instructions, which operate as described herein. In other embodiments, the processors can include controller circuitry, processor circuitry, processors, general purpose single-chip or multi-chip microprocessors, digital signal processors, embedded microprocessors, microcontrollers and the like.

Furthermore, it will be appreciated that in one embodiment, the program logic may advantageously be implemented as one or more components. The components may advantageously be configured to execute on one or more processors. The components include, but are not limited to, software or hardware components, modules such as software modules, object-oriented software components, class components and task components, processes methods, functions, attributes, procedures, subroutines, segments of program code, drivers, firmware, microcode, circuitry, data, databases, data structures, tables, arrays, and variables.

A phrase such as “an aspect” does not imply that such aspect is essential to the subject technology or that such aspect applies to all configurations of the subject technology. A disclosure relating to an aspect may apply to all configurations, or one or more configurations. An aspect may provide one or more examples of the disclosure. A phrase such as “an aspect” may refer to one or more aspects and vice versa. A phrase such as “an embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples of the disclosure. A phrase such “an embodiment” may refer to one or more embodiments and vice versa. A phrase such as “a configuration” does not imply that such configuration is essential to the subject technology or that such configuration applies to all configurations of the subject technology. A disclosure relating to a configuration may apply to all configurations, or one or more configurations. A configuration may provide one or more examples of the disclosure. A phrase such as “a configuration” may refer to one or more configurations and vice versa.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Furthermore, to the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

While certain aspects and embodiments of the subject technology have been described, these have been presented by way of example only, and are not intended to limit the scope of the subject technology. Indeed, the novel methods and systems described herein may be embodied in a variety of other forms without departing from the spirit thereof. The accompanying claims and their equivalents are intended to cover such forms or modifications as would fall within the scope and spirit of the subject technology.

What is claimed is:

1. A method, for characterization of data, comprising:
administering treatment to a patient based on an indicator of a health parameter of a subject, wherein the indicator is determined by,
applying an unfolding algorithm, by a processor, to each of at least two N^{th} order tensors, representing data, to generate at least two matrices, wherein N>2, wherein the at least two tensors have a matching number of columns in each of all dimensions except an N^{th} dimension, wherein the applying the unfolding algorithm preserves the number of columns in one dimension common to (a) one of the at least two tensors and (b) a corresponding one of the at least two matrices, wherein each of the at least two matrices is a full column rank matrix, wherein each of the matrices is a unique, weighted sum of subtensors having a matching number of columns in each of all dimensions, at least two of the sums having different weighting coefficients;
determining a relative significance of the subtensors as a ratio of the weighting coefficients;
determining and outputting, by a processor and based on the relative significance of the subtensors, the indicator of the health parameter of the subject, wherein the health parameter comprises at least one of a differential diagnosis, a first health status of the subject, a disease subtype, at least one of an estimated probability or an estimated risk of a second health status of the subject, a prognosis of the subject, or a predicted response to a treatment of the subject.

2. The method of claim 1, wherein the tensors have one-to-one mappings among the columns across all but the N^{th} dimension of each of the tensors.

3. The method of claim 1, wherein the tensors do not have one-to-one mappings among the rows across the N^{th} dimension of each of the tensors.

4. The method of claim 1, further comprising applying a decomposition algorithm, by a processor, to the at least two subtensors, to generate, from the at least two subtensors A and B, eigenvectors of each of AA^{T}, A^{T}A, BB^{T}, and B^{T}B.

5. The method of claim 1, wherein the data comprises indicators, represented in respective rows and columns of the tensor, of values of at least two index parameters.

6. The method of claim 1, wherein the applying the unfolding algorithm includes appending into (N−1)^{th} order tensors into (N−2)^{th} order tensors that span (N−2) dimensions in each tensor.

7. The method of claim 1, wherein the applying the unfolding algorithm includes appending into a matrix the columns or rows across a preserved dimension in each tensor.

8. The method of claim 1, wherein each subtensor is an outer product of one x-, one y- and one z-axis vector.

9. The method of claim 8, wherein the sets of x-, y- and z-axes vectors are computed by using a matrix GSVD of the tensors unfolded along their corresponding axes.

10. The method of claim 1, wherein administering the treatment comprises administering a drug to the subject, admitting the subject to a care facility, or performing an operation on the subject.

11. The method of claim 1, wherein the tensors are generated by folding a plurality of matrices into the tensors.

12. A method, for characterization of data, comprising:
administering treatment to a patient based on an indicator of a health parameter of a subject,
receiving the indicator of the health parameter of the subject, wherein the health parameter comprises at least one of a differential diagnosis, a first health status of the subject, a disease subtype, at least one of an estimated probability or an estimated risk of a second health status of the subject, a prognosis of the subject, or a predicted response to a treatment of the subject;
wherein the indicator is determined by:
applying an unfolding algorithm, by a processor, to each of at least two N^{th} order tensors, representing data, to generate at least two matrices, wherein N>2, wherein the at least two tensors have a matching number of columns in each of all dimensions except an N^{th} dimension, wherein the applying the unfolding algorithm preserves the number of columns in one dimension common to (a) one of the at least two tensors and (b) a corresponding one of the at least two matrices, wherein each of the at least two matrices is a full column rank matrix, wherein each of the matrices is a unique, weighted sum of subtensors having a matching number of columns in each of all dimensions, at least two of the sums having different weighting coefficients;
determining a relative significance of the subtensors as a ratio of the weighting coefficients;
determining, based on the relative significance of the subtensors, the indicator.

13. The method of claim 12, wherein the treatment comprises administering a drug to the subject, admitting the subject to a care facility, or performing an operation on the subject.

14. A system, for characterization of data, comprising:
an unfolding module configured to apply an unfolding algorithm, by a processor, to each of at least two N^{th} order tensors, representing data, to generate at least two matrices, wherein N>2, wherein the at least two tensors have a matching number of columns in each of all dimensions except an N^{th} dimension, wherein the applying the unfolding algorithm preserves the number of columns in one dimension common to (a) one of the at least two tensors and (b) a corresponding one of the at least two matrices, wherein each of the at least two matrices is a full column rank matrix, wherein each of the matrices is a unique, weighted sum of subtensors having a matching number of columns in each of all dimensions, at least two of the sums having different weighting coefficients;
a first determining module configured to determine a relative significance of the subtensors as a ratio of the weighting coefficients;
a second determining module configured to determine, by a processor and based on the relative significance of the subtensors, an indicator of a health parameter of a subject, the indicator being used to determine whether to administer treatment to the subject, wherein the health parameter comprises at least one of a differential diagnosis, a first health status of the subject, a disease subtype, at least one of an estimated probability or an estimated risk of a second health status of the subject, a prognosis of the subject, or a predicted response to a treatment of the subject;
an outputting module, configured to output the indicator.

15. The system of claim 14, wherein the tensors have one-to-one mappings among the columns across all but the N^{th} dimension of each of the tensors.

16. The system of claim 14, wherein the tensors do not have one-to-one mappings among the rows across the N^{th} dimension of each of the tensors.

17. The system of claim 14, further comprising applying a decomposition algorithm, by a processor, to the at least two subtensors, to generate, from the at least two subtensors A and B, eigenvectors of each of AA^{T}, A^{T}A, BB^{T}, and B^{T}B.

18. The system of claim 14, wherein the data comprises indicators, represented in respective rows and columns of the tensor, of values of at least two index parameters.

19. The system of claim 14, wherein the applying the unfolding algorithm includes appending into (N−1)^{th} order tensors into (N−2)^{th} order tensors that span (N−2) dimensions in each tensor.

20. The system of claim 14, wherein the applying the unfolding algorithm includes appending into a matrix the columns or rows across a preserved dimension in each tensor.

21. The system of claim 14, wherein each subtensor is an outer product of one x-, one y- and one z-axis vector.

22. The system of claim 21, wherein the sets of x-, y- and z-axes vectors are computed by using a matrix GSVD of the tensors unfolded along their corresponding axes.

23. The system of claim 14, wherein administering the treatment comprises administering a drug, admitting the subject to a care facility, or performing an operation on the subject.

24. The system of claim 14, wherein the tensors are generated by folding a plurality of matrices into the tensors.

25. A method, for characterization of data, comprising:
applying an unfolding algorithm, by a processor, to each of at least two N^{th} order tensors, representing data, to generate at least two matrices, wherein N>2, wherein the at least two tensors have a matching number of columns in each of all dimensions except an N^{th} dimension, wherein the applying the unfolding algorithm preserves the number of columns in one dimension common to (a) one of the at least two tensors and (b) a corresponding one of the at least two matrices, wherein each of the at least two matrices is a full column rank matrix, wherein each of the matrices is a unique, weighted sum of subtensors having a matching number of columns in each of all dimensions, at least two of the sums having different weighting coefficients;
determining a relative significance of the subtensors as a ratio of the weighting coefficients;
determining and outputting, by a processor and based on the relative significance of the subtensors, an indicator of a health parameter of a subject, wherein the health parameter comprises at least one of a differential diagnosis, a first health status of the subject, a disease subtype, at least one of an estimated probability or an estimated risk of a second health status of the subject, a prognosis of the subject, or a predicted response to a treatment of the subject.

26. The method of claim 25, wherein the tensors have one-to-one mappings among the columns across all but the N^{th} dimension of each of the tensors.

27. The method of claim 25, wherein the tensors do not have one-to-one mappings among the rows across the N^{th} dimension of each of the tensors.

28. The method of claim 25, further comprising applying a decomposition algorithm, by a processor, to the at least two subtensors, to generate, from the at least two subtensors A and B, eigenvectors of each of AA^{T}, A^{T}A, BB^{T}, and B^{T}B.

29. The method of claim 25, wherein the data comprises indicators, represented in respective rows and columns of the tensor, of values of at least two index parameters.

30. The method of claim 25, wherein the applying the unfolding algorithm includes appending into (N−1)^{th} order tensors into (N−2)^{th} order tensors that span (N−2) dimensions in each tensor.

31. The method of claim 25, wherein the applying the unfolding algorithm includes appending into a matrix the columns or rows across a preserved dimension in each tensor.

32. The method of claim 25, wherein each subtensor is an outer product of one x-, one y- and one z-axis vector.

33. The method of claim 32, wherein the sets of x-, y- and z-axes vectors are computed by using a matrix GSVD of the tensors unfolded along their corresponding axes.

34. The method of claim 25, wherein the tensors are generated by folding a plurality of matrices into the tensors.

35. A system, for characterization of data, comprising:
an unfolding module configured to apply an unfolding algorithm, by a processor, to each of at least two N^{th} order tensors, representing data, to generate at least two matrices, wherein N>2, wherein the at least two tensors have a matching number of columns in each of all dimensions except an N^{th} dimension, wherein the applying the unfolding algorithm preserves the number of columns in one dimension common to (a) one of the at least two tensors and (b) a corresponding one of the at least two matrices, wherein each of the at least two matrices is a full column rank matrix, wherein each of the matrices is a unique, weighted sum of subtensors having a matching number of columns in each of all dimensions, at least two of the sums having different weighting coefficients;
a first determining module configured to determine a relative significance of the subtensors as a ratio of the weighting coefficients;
a second determining module configured to determine, by a processor and based on the relative significance of the subtensors, an indicator of a health parameter of a subject, wherein the health parameter comprises at least one of a differential diagnosis, a first health status of the subject, a disease subtype, at least one of an estimated probability or an estimated risk of a second health status of the subject, a prognosis of the subject, or a predicted response to a treatment of the subject;
an outputting module, configured to output the indicator.

36. The system of claim 35, wherein the tensors have one-to-one mappings among the columns across all but the N^{th} dimension of each of the tensors.

37. The system of claim 35, wherein the tensors do not have one-to-one mappings among the rows across the N^{th} dimension of each of the tensors.

38. The system of claim 35, further comprising applying a decomposition algorithm, by a processor, to the at least two subtensors, to generate, from the at least two subtensors A and B, eigenvectors of each of AA^{T}, A^{T}A, BB^{T}, and B^{T}B.

39. The system of claim 35, wherein the data comprises indicators, represented in respective rows and columns of the tensor, of values of at least two index parameters.

40. The system of claim 35, wherein the applying the unfolding algorithm includes appending into (N−1)^{th} order tensors into (N−2)^{th} order tensors that span (N−2) dimensions in each tensor.

41. The system of claim 35, wherein the applying the unfolding algorithm includes appending into a matrix the columns or rows across a preserved dimension in each tensor.

42. The system of claim 35, wherein each subtensor is an outer product of one x-, one y- and one z-axis vector.

43. The system of claim 42, wherein the sets of x-, y- and z-axes vectors are computed by using a matrix GSVD of the tensors unfolded along their corresponding axes.

44. The system of claim 35, wherein the tensors are generated by folding a plurality of matrices into the tensors.
